(19) United States
(12) Patent Application Publication
(10) Pub. No.: US 2026/0132214 A1
(43) Pub. Date: May 14, 2026

(54) GLYPICAN 3 ANTIBODY AND RELATED METHODS

(71) Applicant: JECHO LABORATORIES, INC., Frederick, MD (US)

(72) Inventors: Jianwei Zhu, Frederick, MD (US); Haiqiu Huang, North Bethesda, MD (US); Zhangyi Song, Rockville, MD (US); Tong Wu, Frederick, MD (US); Shusheng Wang, Ellicott City, MD (US); Yueqing Xie, Clarksburg, MD (US); Hua Jiang, Clarksburg, MD (US)

(21) Appl. No.: 19/170,346

(22) Filed: Apr. 4, 2025

Related U.S. Application Data
(63) Continuation of application No. PCT/US22/77807, filed on Oct. 7, 2022.

(51) Int. Cl. C07K 16/30; A61P 35/00; C07K 16/28

(52) U.S. Cl. CPC C07K 16/303; A61P 35/00; C07K 16/2809; C07K 2317/31; C07K 2317/622; C07K 2317/92

(57) ABSTRACT

The disclosure provides amino acid sequences (e.g., antibodies) that can bind Glypican 3 Protein (GPC3). The disclosure relates to antigen binding fragments of GPC3 antibodies, conjugates, including immunoconjugates, of GPC3 antibodies, bispecific antibodies that include GPC3 antibodies, and related compositions and formulations. The disclosure also relates to methods for using the sequences, e.g., GPC3 antibodies and antigen-binding fragments thereof, in the treatment of GPC3-related diseases, disorders, and conditions including cell proliferative diseases such as cancer, and more particularly, liver cancer.

### RELATED APPLICATIONS

This application is a bypass continuation of co-pending PCT Application No. PCT US2022 077807, filed on Oct. 7, 2022, which is incorporated by reference herein in its entirety.

### INCORPORATION BY REFERENCE

This application incorporates by reference a Sequence Listing submitted with this application in computer readable form (CRF) as an .xml file generated using WIPO Sequence Version 2.1.0 entitled “66348 SequenceListing.xml” created on Oct. 5, 2022 and having a size of 91,683 bytes.

### BACKGROUND

Liver cancer is one of the most common cancers worldwide, with WHO's International Agency for Research on Cancer estimating more than 841,000 new cases (ranked 6th) and more than 781,000 deaths in 2018. Worldwide, Asia accounts for over 72% of the incidences and mortalities, followed by Europe, Africa, and the Americas. Hepatocellular carcinoma (HCC) and cholangiocarcinoma (CCA) are the two major forms of primary liver cancer. According to the American Cancer Society, HCC accounts for ˜75% of all liver cancer cases. The Chinese National Cancer Center estimated that in 2018 HCC accounted for about 84%-92% of liver cancer cases in China. Despite the prevalence of HCC, treatment options remain limited in terms of choices of approved therapeutics, evidence from randomized controlled trials, and survival benefits.

Glypican 3 (GPC3) is highly expressed in most HCC but not in CCA or normal adult tissues. GPC3 is also expressed to a lesser degree in other tumors. It has been reported that GPC3-positive HCC patients have a significantly lower 5-year survival rate than GPC3-negative HCC patients, correlating GPC3 expression with poor clinical prognosis in HCC.

Described herein are specific binding proteins, monoclonal antibodies, and antigen-binding fragments and domains thereof that can bind GPC3 and can be used in methods for detection, treatment, and prophylaxis of GPC3 related disorders, diseases, and associated clinical symptoms, including cancer.

### SUMMARY OF THE DISCLOSURE

The disclosure generally provides an antibody (i.e., isolated antibodies, and antigen-binding fragments thereof), that bind to glypican 3 protein (GPC3). In some aspects, the disclosure provides an antibody, isolated antibodies, and antigen-binding fragments thereof that exhibit specific binding to an epitope of a GPC3 protein. In some embodiments, the antibody, or antigen-binding fragment thereof can comprise one or more heavy chain variable regions, one or more light chain variable regions, one or more CDR1 of a light and/or heavy chain variable region, one or more CDR2 of a light and/or heavy chain variable region, one or more CDR3 of a light and/or heavy chain variable region, or any combinations thereof, according to the aspects and embodiments provided by the disclosure.

In embodiments, the antibody, or antigen-binding fragment thereof binds to an epitope within the sequence of a human GPC3 protein sequence (SEQ ID NO: 84).

Human GPC-3 (P51654-1)
(SEQ ID NO: 84)
MAGTVRTACL VVAMLLSLDF PGQAQPPPPP PDATCHQVRS
FFQRLQPGLK WVPETPVPGS DLQVCLPKGP TCCSRKMEEK
YQLTARLNME QLLQSASMEL KFLIIQNAAV FQEAFEIVVR
HAKNYTNAMF KNNYPSLTPQ AFEFVGEFFT DVSLYILGSD
INVDDMVNEL FDSLFPVIYT QLMNPGLPDS ALDINECLRG
ARRDLKVEGN FPKLIMTQVS KSLQVTRIFL QALNLGIEVI
NTTDHLKESK DCGRMLTRMW YCSYCQGLMM VKPCGGYCNV
VMQGCMAGVV EIDKYWREYI LSLEELVNGM YRIYDMENVL
LGLFSTIHDS IQYVQKNAGK LTTTIGKLCA HSQQRQYRSA
YYPEDLFIDK KVLKVAHVEH EETLSSRRRE LIQKLKSFIS
FYSALPGYIC SHSPVAENDT LCWNGQELVE RYSQKAARNG
MKNQFNLHEL KMKGPEPVVS QIIDKLKHIN QLLRTMSMPK
GRVLDKNLDE EGFESGDCGD DEDECIGGSG DGMIKVKNQL
RFLAELAYDL DVDDAPGNSQ QATPKDNEIS TFHNLGNVHS
PLKLLTSMAI SVVCFFFLVH

In some aspects, the disclosure relates to an isolated antibody or antigen-binding fragment thereof that binds to glypican 3 (GPC3) protein, the antibody or antigen-binding fragment thereof comprising: (i) a heavy chain variable region comprising: a CDR1, a CDR2, and a CDR3 of the amino acid sequence of SEQ ID NO: 1; and a light chain variable region comprising: a CDR1, a CDR2, and a CDR3 amino acid sequences of SEQ ID NO: 2; (ii) a heavy chain variable region comprising: a CDR1, a CDR2, and a CDR3 of the amino acid sequence of SEQ ID NO: 3; and a light chain variable region comprising: a CDR1, a CDR2, and a CDR3 amino acid sequences of SEQ ID NO: 4; (iii) a heavy chain variable region comprising: a CDR1, a CDR2, and a CDR3 of the amino acid sequence of SEQ ID NO: 5; and a light chain variable region comprising: a CDR1, a CDR2, and a CDR3 amino acid sequences of SEQ ID NO: 6; (iv) a heavy chain variable region comprising: a CDR1, a CDR2, and a CDR3 of the amino acid sequence of SEQ ID NO: 7; and a light chain variable region comprising: a CDR1, a CDR2, and a CDR3 amino acid sequences of SEQ ID NO: 8; (v) a heavy chain variable region comprising: a CDR1, a CDR2, and a CDR3 of the amino acid sequence of SEQ ID NO: 9; and a light chain variable region comprising: a CDR1, a CDR2, and a CDR3 amino acid sequences of SEQ ID NO: 10; (vi) a heavy chain variable region comprising: a CDR1, a CDR2, and a CDR3 of the amino acid sequence of SEQ ID NO: 11; and a light chain variable region comprising: a CDR1, a CDR2, and a CDR3 amino acid sequences of SEQ ID NO: 12; (vii) a heavy chain variable region comprising: a CDR1, a CDR2, and a CDR3 of the amino acid sequence of SEQ ID NO: 13; and a light chain variable region comprising: a CDR1, a CDR2, and a CDR3 amino acid sequences of SEQ ID NO: 14; (viii) a heavy chain variable region comprising: a CDR1, a CDR2, and a CDR3 of the amino acid sequence of SEQ ID NO: 15; and a light chain variable region comprising: a CDR1, a CDR2, and a CDR3 amino acid sequences of SEQ ID NO: 16; (ix) a heavy chain variable region comprising: a CDR1, a CDR2, and a CDR3 of the amino acid sequence of SEQ ID NO: 48; and a light chain variable region comprising: a CDR1, a CDR2, and a CDR3 amino acid sequences of SEQ ID NO: 8; (xi) a heavy chain variable region comprising: a CDR1, a CDR2, and a CDR3 of the amino acid sequence of SEQ ID NO: 52; and a light chain variable region comprising: a CDR1, a CDR2, and a CDR3 amino acid sequences of SEQ ID NO: 8; (xii) a heavy chain variable region comprising: a CDR1, a CDR2, and a CDR3 of the amino acid sequence of SEQ ID NO: 56; and a light chain variable region comprising: a CDR1, a CDR2, and a CDR3 amino acid sequences of SEQ ID NO: 8; (xiii) a heavy chain variable region comprising: a CDR1, a CDR2, and a CDR3 of the amino acid sequence of SEQ ID NO: 60; and a light chain variable region comprising: a CDR1, a CDR2, and a CDR3 amino acid sequences of SEQ ID NO: 8; (xiv) a heavy chain variable region comprising: a CDR1, a CDR2, and a CDR3 of the amino acid sequence of SEQ ID NO: 64; and a light chain variable region comprising: a CDR1, a CDR2, and a CDR3 amino acid sequences of SEQ ID NO: 8; (xv) a heavy chain variable region comprising: a CDR1, a CDR2, and a CDR3 of the amino acid sequence of SEQ ID NO: 68; and a light chain variable region comprising: a CDR1, a CDR2, and a CDR3 amino acid sequences of SEQ ID NO: 8; (xvi) a heavy chain variable region comprising: a CDR1, a CDR2, and a CDR3 of the amino acid sequence of SEQ ID NO: 72; and a light chain variable region comprising: a CDR1, a CDR2, and a CDR3 amino acid sequences of SEQ ID NO: 8; (xvii) a heavy chain variable region comprising: a CDR1, a CDR2, and a CDR3 of the amino acid sequence of SEQ ID NO: 76; and a light chain variable region comprising: a CDR1, a CDR2, and a CDR3 amino acid sequences of SEQ ID NO: 8; or (xviii) a heavy chain variable region comprising: a CDR1, a CDR2, and a CDR3 of the amino acid sequence of SEQ ID NO: 80; and a light chain variable region comprising: a CDR1, a CDR2, and a CDR3 amino acid sequences of SEQ ID NO: 8.

In some embodiments of this aspect, the disclosure provides an isolated antibody or antigen-binding fragment thereof of claim 1, comprising: (i) a heavy chain variable region of SEQ ID NO: 1; and a light chain variable region of SEQ ID NO: 2; (ii) a heavy chain variable region of SEQ ID NO: 3; and a light chain variable region of SEQ ID NO: 4; (iii) a heavy chain variable region of SEQ ID NO: 5; and a light chain variable region of SEQ ID NO: 6; (iv) a heavy chain variable region of SEQ ID NO: 7; and a light chain variable region of SEQ ID NO: 8; (v) a heavy chain variable region of SEQ ID NO: 9; and a light chain variable region of SEQ ID NO: 10; (vi) a heavy chain variable region of SEQ ID NO: 11; and a light chain variable region of SEQ ID NO: 12; (vii) a heavy chain variable region of SEQ ID NO: 13; and a light chain variable region of SEQ ID NO: 14; (viii) a heavy chain variable region of SEQ ID NO: 15; and a light chain variable region of SEQ ID NO: 16; (ix) a heavy chain variable region of SEQ ID NO: 48; and a light chain variable region of SEQ ID NO: 8; (xi) a heavy chain variable region of SEQ ID NO: 52; and a light chain variable region of SEQ ID NO: 8; (xii) a heavy chain variable region of SEQ ID NO: 56; and a light chain variable region of SEQ ID NO: 8; (xiii) a heavy chain variable region of SEQ ID NO: 60; and a light chain variable region of SEQ ID NO: 8; (xiv) a heavy chain variable region of SEQ ID NO: 64; and a light chain variable region of SEQ ID NO: 8; (xv) a heavy chain variable region of SEQ ID NO: 68; and a light chain variable region of SEQ ID NO: 8; (xvi) a heavy chain variable region of SEQ ID NO: 72; and a light chain variable region of SEQ ID NO: 8; (xvii) a heavy chain variable region of SEQ ID NO: 76; and a light chain variable region of SEQ ID NO: 8; or (xviii) a heavy chain variable region of SEQ ID NO: 80; and a light chain variable region of SEQ ID NO: 8.

In some embodiments of the above aspects, the disclosure provides an antigen-binding fragment comprising a single-chain Fv (scFv), a single chain Fv-Fc (scFv-Fc), a single-chain antibody, a single domain antibody, a Fab fragment, or a F(ab′)2 fragment.

In some embodiments of the above aspects, the disclosure provides an antibody comprising an IgG, an IgM, an IgA, an IgE, an IgD, or an IgY isotype.

In some embodiments of the above aspects, the disclosure provides a monoclonal antibody (mAb).

In some embodiments of the above aspects, the disclosure provides an antibody or an antigen-binding fragment that is conjugated to a moiety. In some embodiments, the conjugated moiety comprises a therapeutic agent, an active agent, a solid support, an affinity agent, or a detectable label. In some further embodiments, the moiety comprises a cytotoxin. In some other embodiments, the moiety comprises an anti-cancer agent.

In some embodiments of the above aspects, the antibody or an antigen-binding fragment thereof binds to the glypican 3 protein with a K_{D} of from about 1.0 pM to 200 nM.

In some embodiments of the above aspects, the antibody or antigen-binding fragment thereof inhibits cell proliferation with an IC50 of from about 0.01-250 nM.

In another aspect, the disclosure provides a bispecific antibody that comprising an antibody or antigen-binding fragment thereof in accordance with any of the above aspects and embodiments, and an antibody or antigen-binding fragment thereof that specifically binds to an antigen that does not comprise a GPC3 epitope. In some further embodiments, the bispecific antibody comprises a GPC3 antibody and a therapeutic antibody. In further embodiments, the bispecific antibody comprises an antibody or antigen-binding fragment thereof that specifically binds to an antigen that does not comprise a GPC3 epitope and induces an immune response. In yet further embodiments, the bispecific antibody comprises a GPC3 antibody or fragment thereof in accordance with the disclosure and antibody or antigen-binding fragment thereof that specifically binds to an antigen comprising CD2, CD3, CD11a CD20, CD25 (IL2R), CD33, CD52, EGFR, VEGF, Integrin-alpha 3, GPIIb/Illar, Protein F, TNF-alpha, TNF-beta, HER2/Neu, C5, or IgE.

In any of the above aspects and embodiments, the isolated antibody or antigen-binding fragment thereof can be conjugated to a solid support, an affinity agent, or a detectable agent.

In another aspect, the disclosure provides a pharmaceutical composition comprising the isolated antibody or antigen-binding fragment thereof according to any of the above aspect and embodiments and a pharmaceutically acceptable carrier.

In another aspect, the disclosure provides a kit comprising the pharmaceutical composition or the antibody or antigen-binding fragment thereof according to any of the above aspects and embodiments, optional reagents, and instructions for use.

In another aspect, the disclosure provides a method for treating cancer in a subject in need thereof, the method comprising administering to the subject an effective amount of an antibody or antigen-binding fragment thereof that binds to glypican 3 (GPC3) protein, the antibody or antigen-binding fragment thereof comprising: comprising: (i) a heavy chain variable region comprising: a CDR1, a CDR2, and a CDR3 of the amino acid sequence of SEQ ID NO: 1; and a light chain variable region comprising: a CDR1, a CDR2, and a CDR3 amino acid sequences of SEQ ID NO: 2; (ii) a heavy chain variable region comprising: a CDR1, a CDR2, and a CDR3 of the amino acid sequence of SEQ ID NO: 3; and a light chain variable region comprising: a CDR1, a CDR2, and a CDR3 amino acid sequences of SEQ ID NO: 4; (iii) a heavy chain variable region comprising: a CDR1, a CDR2, and a CDR3 of the amino acid sequence of SEQ ID NO: 5; and a light chain variable region comprising: a CDR1, a CDR2, and a CDR3 amino acid sequences of SEQ ID NO: 6; (iv) a heavy chain variable region comprising: a CDR1, a CDR2, and a CDR3 of the amino acid sequence of SEQ ID NO: 7; and a light chain variable region comprising: a CDR1, a CDR2, and a CDR3 amino acid sequences of SEQ ID NO: 8; (v) a heavy chain variable region comprising: a CDR1, a CDR2, and a CDR3 of the amino acid sequence of SEQ ID NO: 9; and a light chain variable region comprising: a CDR1, a CDR2, and a CDR3 amino acid sequences of SEQ ID NO: 10; (vi) a heavy chain variable region comprising: a CDR1, a CDR2, and a CDR3 of the amino acid sequence of SEQ ID NO: 11; and a light chain variable region comprising: a CDR1, a CDR2, and a CDR3 amino acid sequences of SEQ ID NO: 12; (vii) a heavy chain variable region comprising: a CDR1, a CDR2, and a CDR3 of the amino acid sequence of SEQ ID NO: 13; and a light chain variable region comprising: a CDR1, a CDR2, and a CDR3 amino acid sequences of SEQ ID NO: 14; (viii) a heavy chain variable region comprising: a CDR1, a CDR2, and a CDR3 of the amino acid sequence of SEQ ID NO: 15; and a light chain variable region comprising: a CDR1, a CDR2, and a CDR3 amino acid sequences of SEQ ID NO: 16; (ix) a heavy chain variable region comprising: a CDR1, a CDR2, and a CDR3 of the amino acid sequence of SEQ ID NO: 48; and a light chain variable region comprising: a CDR1, a CDR2, and a CDR3 amino acid sequences of SEQ ID NO: 8; (xi) a heavy chain variable region comprising: a CDR1, a CDR2, and a CDR3 of the amino acid sequence of SEQ ID NO: 52; and a light chain variable region comprising: a CDR1, a CDR2, and a CDR3 amino acid sequences of SEQ ID NO: 8; (xii) a heavy chain variable region comprising: a CDR1, a CDR2, and a CDR3 of the amino acid sequence of SEQ ID NO: 56; and a light chain variable region comprising: a CDR1, a CDR2, and a CDR3 amino acid sequences of SEQ ID NO: 8; (xiii) a heavy chain variable region comprising: a CDR1, a CDR2, and a CDR3 of the amino acid sequence of SEQ ID NO: 60; and a light chain variable region comprising: a CDR1, a CDR2, and a CDR3 amino acid sequences of SEQ ID NO: 8; (xiv) a heavy chain variable region comprising: a CDR1, a CDR2, and a CDR3 of the amino acid sequence of SEQ ID NO: 64; and a light chain variable region comprising: a CDR1, a CDR2, and a CDR3 amino acid sequences of SEQ ID NO: 8; (xv) a heavy chain variable region comprising: a CDR1, a CDR2, and a CDR3 of the amino acid sequence of SEQ ID NO: 68; and a light chain variable region comprising: a CDR1, a CDR2, and a CDR3 amino acid sequences of SEQ ID NO: 8; (xvi) a heavy chain variable region comprising: a CDR1, a CDR2, and a CDR3 of the amino acid sequence of SEQ ID NO: 72; and a light chain variable region comprising: a CDR1, a CDR2, and a CDR3 amino acid sequences of SEQ ID NO: 8; (xvii) a heavy chain variable region comprising: a CDR1, a CDR2, and a CDR3 of the amino acid sequence of SEQ ID NO: 76; and a light chain variable region comprising: a CDR1, a CDR2, and a CDR3 amino acid sequences of SEQ ID NO: 8; or (xviii) a heavy chain variable region comprising: a CDR1, a CDR2, and a CDR3 of the amino acid sequence of SEQ ID NO: 80; and a light chain variable region comprising: a CDR1, a CDR2, and a CDR3 amino acid sequences of SEQ ID NO: 8.

In some further embodiments of the above aspect, the method comprises administering an antibody or antigen-binding fragment thereof comprising a heavy chain variable region comprising SEQ ID NO: 1 and a light chain variable region comprising SEQ ID NO: 2; a heavy chain variable region comprising SEQ ID NO: 3 and a light chain variable region comprising SEQ ID NO: 4; a heavy chain variable region comprising SEQ ID NO: 5 and a light chain variable region comprising SEQ ID NO: 6; a heavy chain variable region comprising SEQ ID NO: 7 and a light chain variable region comprising SEQ ID NO: 8; a heavy chain variable region comprising SEQ ID NO: 9 and a light chain variable region comprising SEQ ID NO: 10; a heavy chain variable region comprising SEQ ID NO: 11 and a light chain variable region comprising SEQ ID NO: 12; a heavy chain variable region comprising SEQ ID NO: 13 and a light chain variable region comprising SEQ ID NO: 14; a heavy chain variable region comprising SEQ ID NO: 15 and a light chain variable region comprising SEQ ID NO: 16; a heavy chain variable region comprising SEQ ID NO: 48 and a light chain variable region comprising SEQ ID NO: 8; a heavy chain variable region comprising SEQ ID NO: 52 and a light chain variable region comprising SEQ ID NO: 8; a heavy chain variable region comprising SEQ ID NO: 56 and a light chain variable region comprising SEQ ID NO: 8; a heavy chain variable region comprising SEQ ID NO: 60 and a light chain variable region comprising SEQ ID NO: 8; a heavy chain variable region comprising SEQ ID NO: 64 and a light chain variable region comprising SEQ ID NO: 8; a heavy chain variable region comprising SEQ ID NO: 68 and a light chain variable region comprising SEQ ID NO: 8; a heavy chain variable region comprising SEQ ID NO: 72 and a light chain variable region comprising SEQ ID NO: 8; a heavy chain variable region comprising SEQ ID NO: 76 and a light chain variable region comprising SEQ ID NO: 8; or a heavy chain variable region comprising SEQ ID NO: 80 and a light chain variable region comprising SEQ ID NO: 8.

In some embodiments of the above methods, wherein the cancer comprises a solid tumor cell cancer. In some further embodiments, the cancer comprises a liver cancer.

In another aspect, the disclosure provides a method of inhibiting proliferation of a cell that expresses glypican 3 (GPC3) protein, the method comprising contacting the cell with an effective amount of an antibody or antigen-binding fragment thereof according to the above aspects and embodiments. In some embodiments of this method, the cell comprises a cancer cell. In some further embodiments of this method, the cell comprises a liver cancer cell.

In another aspect, the disclosure provides a method of detecting the presence of glypican 3 (GPC3) protein in a biological sample, comprising contacting the biological sample with an effective amount of an antibody or antigen-binding fragment thereof according to the above aspects and embodiments under conditions that allow for formation of a complex between the antibody or antigen-binding fragment thereof and GPC3 present in the sample, and measuring a detectable signal associated with the formation of the complex. In further embodiments of these methods, the antibody or antigen-binding fragment thereof comprises a detectable label.

In any of the above aspects and embodiments relating to methods, some embodiments provide for a subject who is a human, and/or a cell that is a human cell, and/or a biological sample that is from a human.

In another aspect, the disclosure provides an isolated polynucleotide encoding an antibody or antigen-binding fragment thereof according to the above aspects and embodiments.

In another aspect, the disclosure provides an isolated recombinant cell that produces an antibody or antigen-binding fragment thereof according to the above aspects and embodiments.

Other aspects and embodiments of the disclosure will be apparent to one of skill in the art in light of the disclosure and illustrative examples that follow.

### BRIEF DESCRIPTION OF THE DRAWINGS

FIG. 1 depicts cell surface binding activities of the GPC3 antibody constructs in accordance with example embodiments of the disclosure in cells that express GPC3 at the cell surface (HepG2) and cells that do not express GPC3 at the cell surface (NCI1975; H929).

FIG. 2 depicts cell internalization profiles of the GPC3 antibody constructs in accordance with example embodiments of the disclosure, in HepG2 cells (2A) and Huh7 cells (2B).

FIG. 3 depicts cell proliferation inhibitory activity of the GPC3 antibody construct conjugates in accordance with example embodiments of the disclosure, in HepG2 cells (3A) and Huh7 cells (3B).

FIG. 4 depicts cell apoptosis activity of the GPC3 antibody construct conjugates in accordance with example embodiments of the disclosure, in HepG2 cells.

FIG. 5 depicts calculated score associated with a heavy chain variable sequence region in accordance with the disclosure and identifies probability of an amino acid residue interaction with a GPC3 epitope.

FIG. 6 depicts binding kinetics of affinity matured GPC3 antibody constructs in accordance with example embodiments of the disclosure.

FIG. 7 depicts ELISA curves of affinity matured GPC3 antibody constructs in accordance with example embodiments of the disclosure.

FIG. 8 depicts cell surface binding activities of affinity matured GPC3 antibody constructs in accordance with example embodiments of the disclosure.

FIG. 9 depicts cell surface binding activity of affinity matured GPC3 antibody constructs in accordance with example embodiments of the disclosure, and that the constructs lack non-specific binding activity.

### DETAILED DESCRIPTION

It is to be understood that this disclosure is not limited to specific proteins, nucleic acids, compositions, methods or process steps that are described below as exemplary and illustrative aspects and embodiments, as variations and modifications fall within the scope of the description.

As used in this specification and the appended claims, the singular form “a”, “an” and “the” include plural referents unless the context clearly dictates otherwise. Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this invention. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### A. Antibodies

As used herein, the term “antibody” or its plural, “antibodies”, also known as immunoglobulins, encompass full-length antibody sequences including, for example, monoclonal antibodies (including full-length monoclonal antibodies), polyclonal antibodies, multispecific antibodies formed from at least two different epitope binding fragments, bispecific antibodies, human antibodies, and humanized antibodies. Antibody “fragments” (or “antigen-binding fragments”, “binding fragments”, “epitope-binding fragments”, and the like) as described herein typically refer to any antibody sequence that is less than the full-length antibody sequence, and still exhibits specific binding activity to the target antigen. In example embodiments, antibody fragments typically comprise at least a combination of three CDR sequences of a heavy chain variable domain (HCDR1, HCDR2, HCDR3) and at least three CDR sequences of a light chain variable domain (LCDR1, LCDR2, LCDR3). Some non-limiting examples of antibody fragments include single-chain Fvs (scFv), single chain Fv-Fc (scFv-Fc), single-chain antibodies, single domain antibodies, domain antibodies, Fab fragments, F(ab′)2 fragments, camelised antibodies, antibody fragments that exhibit the desired biological activity (e.g. the antigen binding portion), disulfide-linked Fvs (dsFv), and anti-idiotypic (anti-Id) antibodies, intrabodies, and epitope-binding fragments of any of the above. In some embodiments, the disclosure provides antibodies that include immunoglobulin molecules and immunologically active fragments of immunoglobulin molecules, i.e., molecules that contain at least one antigen-binding site. Antibodies and fragments thereof may also include peptide fusions with antibodies or portions thereof such as a protein fused to an Fc domain.

Immunoglobulin molecules can be of any isotype (e.g., IgG, IgE, IgM, IgD, IgA and IgY), subisotype (e.g., IgG1, IgG2, IgG3, IgG4, IgAQ1 and IgA2) or allotype (e.g., Gm, e.g., Glm (f, z, a or x), G2m(n), G3m (g, b, or c), Am, Em, and Km (1, 2 or 3)). Antibodies and fragments thereof may be derived from any mammal, including, but not limited to, humans, monkeys, pigs, horses, rabbits, dogs, cats, mice, etc., or other animals such as birds (e.g. chickens).

In some aspects, the disclosure provides novel binding agents. In some exemplary embodiments, the novel binding proteins are IgGs, scFvs, Fab, monoclonal antibodies (mAbs) or single chain Fv-Fc (scFv-Fc) antibodies. A typical or conventional mAb comprises two heavy chain subunits and two light chain subunits. Each mAb heavy chain contains one variable domain (VH) which contributes to antigen binding and a constant domain (CH) made up of three or four subregions (C_{H}1, C_{H}2, C_{H}3, C_{H}4). The VH comprises three complementarity-determining regions (CDRs), HCDR1, HCDR2, and HCDR3. Each mAb light chain contains one variable domain (VL) and one constant domain (CL). The VL comprises three CDRs, LCDR1, LCDR2, and LCDR3. There are two isotypes of light chain constant domains, kappa (κ) and lambda (λ), found in mammals. Disulfide bonds join each CHI domain to one CL domain, and join C_{H}2 domains to one another. Five types of heavy chains (α, δ, ε, γ, and μ) are found in different classes of antibodies (IgA, IgD, IgE, IgG, and IgM). mAb heavy chains have hinge regions that confer structural flexibility and mobility.

The “Fc” region encompasses domains from the constant region of the heavy chain of an immunoglobulin, including a fragment, analog, variant, mutant, or derivative thereof. Suitable immunoglobulins include IgG1, IgG2, IgG3, IgG4, and other classes such as IgA, IgD, IgE and IgM. The Fc region may be a native sequence Fc region or an altered Fc region. The Fc region of an immunoglobulin generally comprises two constant domains, a C_{H}2 domain and a C_{H}3 domain. The “Fv” region encompasses the VH and VL domains of an immunoglobulin. As used herein, “scFv-Fc” antibodies refer to a fusion protein of a single VH and a single VL domain, connected with a hinge region, and the C_{H}2 domain and the C_{H}3 domain of a single C_{H} domain.

As discussed herein, the antibodies and antigen-binding fragments thereof disclosed herein comprise binding domains that bind to an epitope of GPC3. “Binding domain” or “binding sequence” may be interchangeably used with an antibody fragment or “antigen-binding fragment”, and as used herein refers to a portion of an antibody sequence that is adequate and sufficient to bind to or to interact with a target structure, antigen, or epitope. In some aspects, the antibodies bind to a region of GPC3 that comprises an antigenic sequence or fragment of SEQ ID NO: 84. In other aspects, the antibodies bind to a domain of the GPC3 protein. For example, in embodiments the binding domain can bind specifically to an epitope within the N-terminal subunit of GPC3, having a molecular weight of about 40 kDa. In some embodiments the binding domain can bind specifically to an epitope within the C-terminal subunit of GPC3, having a molecular weight of about 30 kDa.

In some embodiments the antibodies and antigen-binding fragments thereof in accordance with the disclosure bind to GPC3, and can inhibit GPC3 binding interactions with other molecules. In some example embodiments the antibodies and antigen-binding fragments thereof can inhibit or antagonize one or more GPC3-assoicated signaling pathways. Non-limiting examples of such activity include cellular pathways involved with cell division, cell growth regulation, Wnt/beta-catenin signaling, frizzled signaling, protocadherin FAT1 signaling, or Yap signaling. In accordance with such example embodiments of the disclosure, the antibodies or fragments thereof can prevent, inhibit, or reduce binding of and/or activity of GPC3, and may provide neutralizing activity against GPC3 as well as its downstream signaling partners and pathways.

In some embodiments, the antibodies disclosed herein can be characterized by one or more of the following structural and/or functional properties:
a. an amino acid sequence comprising a VH domain and a VL domain, wherein the VH domain comprises an HCDR1, HCDR2, and HCDR3 identified in SEQ ID NOs: 1, 3, 5, 7, 9, 11, 13, 15, 48, 52, 56, 60, 64, 68, 72, 76, or 80; and wherein the VL domain comprises a LCDR1, a LCDR2, and an LCDR3 identified in SEQ ID NOs: 2, 4, 6, 8, 10, 12, 14, or 16;
b. an amino acid sequence comprising a VH domain having the amino acid sequence of SEQ ID NOs: 1, 3, 5, 7, 9, 11, 13, 15, 48, 52, 56, 60, 64, 68, 72, 76, or 80 and/or a VL domain having the amino acid sequence of SEQ ID NOs: 2, 4, 6, 8, 10, 12, 14, or 16;
c. an amino acid sequence having an HCDR1 comprising SEQ ID NOs: 85, 86, 97, 49, 53, 57, 61, 65, 69, 73, 77, or 81; an HCDR2 comprising SEQ ID NOs: 50, 54, 58, 62, 66, 70, 74, 78, 82, 87, 88, or 98; an HCDR3 comprising SEQ ID NOs: 51, 55, 59, 63, 67, 71, 75, 79, 83, 89, 90, 91, 99, 100, or 101; and/or an LCDR1 comprising SEQ ID NOs: 93; an LCDR 2 comprising SEQ ID NOs: 94; an LCDR2 comprising SEQ ID NOs: 95 or 96.
d. binding specificity for GPC3;
e. regarding GPC3, a dissociation constant (K_{D}) in the range of 1 pM-500 nM, 10 pM-500 nM, 0.1 nM-500 nM, 1-450 nM, 1-400 nM, 1-300 nM, 1-200 nM, 1-100 nM, 1-50 nM, 1-25 nM, or 1-10 nM;
f. regarding GPC3, an IC50 value (for in the range of 0.01-250 nM, 0.01-200 nM, 0.01-150 nM, 0.01-100 nM, 0.01-50 nM, 0.01-25 nM, 0.01-20 nM, or 0.01-10 nM;
g. the ability to inhibit, reduce, prevent, or disrupt GPC3 binding interactions, as may be measured by an assay including, for example, filter binding assay, fluorescence spectroscopy, ELISA, isothermal titration calorimetry, and surface plasma resonance.

In some aspects, an antibody or antigen-binding fragment thereof capable of inhibiting, reducing, preventing, or disrupting the binding activity or biological activity of GPC3, and/or capable of neutralizing the activity of the GPC3, binds to an epitope present in the GPC3 protein. In some example embodiments, the epitope bound by the antibody or fragment thereof may be in the C-terminal domain. In some other embodiments, the epitope bound by the antibody or fragment thereof may be in the N-terminal domain.

In some aspects, an antibody that binds GPC3, or an antigen-binding fragment thereof, comprises a VH domain having the amino acid sequence of SEQ ID NOs: 48, 52, 56, 60, 64, 68, 72, 76, or 80.

In some aspects, an antibody that binds GPC3, or an antigen-binding fragment thereof, comprises a VL domain having the amino acid sequence of SEQ ID NOs: 8.

In certain embodiments of these aspects, an antibody that binds GPC3, or an antigen-binding fragment thereof, comprises a VH domain and VL domain combination selected from the following:
a heavy chain variable region comprising SEQ ID NO: 1; and a light chain variable region comprising SEQ ID NO: 2;
a heavy chain variable region comprising SEQ ID NO: 3; and a light chain variable region comprising SEQ ID NO: 4;
a heavy chain variable region comprising SEQ ID NO: 5; and a light chain variable region comprising SEQ ID NO: 6;
a heavy chain variable region comprising SEQ ID NO: 7; and a light chain variable region comprising SEQ ID NO: 8;
a heavy chain variable region comprising SEQ ID NO: 9; and a light chain variable region comprising SEQ ID NO: 10;
a heavy chain variable region comprising SEQ ID NO: 11; and a light chain variable region comprising SEQ ID NO: 12;
a heavy chain variable region comprising SEQ ID NO: 13; and a light chain variable region comprising SEQ ID NO: 14;
a heavy chain variable region comprising SEQ ID NO: 15; and a light chain variable region comprising SEQ ID NO: 16;
a heavy chain variable region comprising SEQ ID NO: 48; and a light chain variable region comprising SEQ ID NO: 8;
a heavy chain variable region comprising SEQ ID NO: 52; and a light chain variable region comprising SEQ ID NO: 8;
a heavy chain variable region comprising SEQ ID NO: 56; and a light chain variable region comprising SEQ ID NO: 8;
a heavy chain variable region comprising SEQ ID NO: 60; and a light chain variable region comprising SEQ ID NO: 8;
a heavy chain variable region comprising SEQ ID NO: 64; and a light chain variable region comprising SEQ ID NO: 8;
a heavy chain variable region comprising SEQ ID NO: 68; and a light chain variable region comprising SEQ ID NO: 8;
a heavy chain variable region comprising SEQ ID NO: 72; and a light chain variable region comprising SEQ ID NO: 8;
a heavy chain variable region comprising SEQ ID NO: 76; and a light chain variable region comprising SEQ ID NO: 8; or a heavy chain variable region comprising SEQ ID NO: 80; and a light chain variable region comprising SEQ ID NO: 8.

In certain embodiments of these aspects, an antibody, or an antigen-binding fragment thereof, comprises a VH domain and VL domain having the combinations of heavy chain and light chains, or heavy and light chain CDR sequences that are identified in Table 1.

In exemplary embodiments, the antibodies disclosed herein exhibit binding specificity for the GPC3 protein, and can inhibit, reduce, prevent, or disrupt GPC3 binding interactions.

As one of ordinary skill can appreciate, the sequences disclosed herein can be modified to some degree without compromising the ability of the antibody or fragment thereof to interact with the target protein, i.e., GPC3. In some aspects, antibody sequence variants retain the ability to interact with GPC3 such that the binding interaction between GPC3 and any one or more of its binding partners is disrupted, prevented, reduced, or inhibited.

As used herein, sequence “variants” refer to an antibody amino acid sequence comprising at least one amino acid insertion, deletion, and/or substitution, wherein the resulting antibody maintains one or more of its functional characteristics as described herein. In embodiments, a sequence variant maintains the ability to specifically bind GPC3 protein. An amino acid insertion variant is characterized by the insertion of one or more amino acids between two existing amino acids. An amino acid deletion variant is characterized by the deletion of one or more amino acids from the antibody sequence. An amino acid substitution is characterized by at least one amino acid in the sequence being replaced by another amino acid. In embodiments relating to substitutions, the amino acid substitution(s) may be a conservative substitution, (i.e. an amino acid from one family of amino acids (acidic, basic, non-polar, and uncharged, based on side chain characteristics, including size) is substituted with an amino acid from the same family).

In some aspects, the sequence identity between the variant antibody sequence and the antibody sequences disclosed herein will be at least 85%, 86%, 87%, 88%, 89%, 90%, 91%, 92%, 93%, 94%, 95%, 96%, 97%, 98%, or 99%. “Sequence identity” refers to the percentage of amino acid residues that are identical to the sequences being compared.

### B. Labels, Conjugates, and Moieties

The binding proteins, antibodies, or antigen binding fragments thereof disclosed herein can be conjugated to a therapeutic agent, solid support, affinity agent, or a detectable agent. For example, the amino acid sequences disclosed herein may be conjugated to labels for the purposes of diagnostics and other assays wherein the amino acid sequence (i.e., antibody and/or its associated targets(s)) may be detected. Labels include, without limitation, chromophores, fluorophores, fluorescent proteins, phosphorescent dyes, tandem dyes, particles (micro- and nano-particles of various materials (polymer, magnetic, etc.), haptens, enzymes, peptides, and radioisotopes, among others, and combinations thereof.

In certain aspects, the antibodies are conjugated to a fluorophore. The choice of the fluorophore attached to the antibody will determine the absorption and fluorescence emission properties of the conjugated antibody. Physical properties of a fluorophore label that can be used for an antibody and antibody-bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. Other desirable properties of the fluorescent label may include cell permeability and low toxicity, for example if labeling of the antibody is to be performed in a cell or an organism (e.g., a living animal).

In certain aspects, the conjugated label may comprise an enzyme. Enzymes are desirable labels in some embodiments because amplification of the detectable signal can be obtained and result in increased assay sensitivity. The enzyme itself does not produce a detectable response but functions to break down a substrate when it is contacted by an appropriate substrate such that the converted substrate produces a fluorescent, colorimetric or luminescent signal. Enzymes may amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art and include for example, oxidoreductases such as horseradish peroxidase and a substrate such as 3,3′-diaminobenzidine (DAB); phosphatase enzymes such as an acid phosphatase, alkaline and a substrate such as 5-bromo-6-chloro-3-indolyl phosphate (BCIP); glycosidases, such as beta-galactosidase, beta-glucuronidase or beta-glucosidase and a substrate such as 5-bromo-4-chloro-3-indolyl beta-D-galactopyranoside (X-gal); additional enzymes include hydrolases such as cholinesterases and peptidases, oxidases such as glucose oxidase and cytochrome oxidases, and reductases for which suitable substrates are known.

Enzymes and their appropriate substrates that produce chemiluminescence are suitable for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another aspect, haptens, such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain aspects, fluorescent proteins may be conjugated to the antibody as a label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra.

In certain aspects, the label is a radioactive isotope. Examples of suitable radioactive materials include, but are not limited to, iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mln,), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³SM, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh and ⁹⁷Ru.

In some aspects, drugs may be conjugated to the antibody. For example, an antibody may be conjugated to a therapeutic moiety or agent, such as an anti-proliferative, anti-cancer, cytotoxin, or antiviral drug. In some embodiments, the antibody may bind to a target antigen, and the drug (e.g., cancer drug) acts to inhibit, inactivate, or kill a cancer cell. Accordingly, any drug known in the art may be conjugated to an antibody, or fragment thereof, in accordance with the disclosure. In certain embodiments, drugs and other molecules may be conjugated to an antibody via site-specific conjugation.

### C. Polynucleotides Encoding Antibodies & Recombinant Cells Producing Antibodies

The disclosure provides methods for producing antibodies and antibody fragments. In certain aspects, the disclosure provides for recombinant methods of generating the antibodies and/or fragments thereof. In some embodiments, recombinant nucleic acids encoding for the antibody or fragment thereof may be operably linked to one or more regulatory nucleotide sequences in an expression construct. In some embodiments, the nucleic acid sequences encoding the antibody light and heavy chains can be cloned in the same expression vector in any orientation (e.g., light chain in front of the heavy chain or vice versa) or can be cloned in two different vectors. If expression is carried out using one vector, the two coding genes can have their own genetic elements (e.g., promoter, RBS, leader, stop, poly A, etc.) or they can be cloned with one single set of genetic elements, but connected with a cistron element. Regulatory nucleotide sequences may be appropriate for a host cell used for expression. Numerous types of appropriate expression vectors and suitable regulatory sequences are known in the art for a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Any known constitutive or inducible promoters are contemplated for use with the aspects and embodiments included in the disclosure. The promoters may be either naturally occurring promoters, or hybrid promoters that combine elements of more than one promoter. An expression construct may be present in a cell on an episome, such as a plasmid, or the expression construct may be inserted in a chromosome.

In certain aspects, an expression vector contains a selectable marker gene to allow the selection of transformed host cells. Selectable marker genes are known and may vary with the host cell used. In certain aspects, the disclosure relates to an expression vector comprising a nucleotide sequence encoding a polypeptide that is operably linked to at least one regulatory sequence. Regulatory sequences are generally known and may be selected to direct expression of the encoded polypeptide. Accordingly, the term regulatory sequence includes promoters, enhancers, and other expression control elements. Exemplary, non-limiting regulatory sequences are described in Goeddel; Gene Expression Technology: Methods in Enzymology, Academic Press, San Diego, CA (1990). It should be understood that the design of the expression vector may depend on such factors as the choice of the host cell to be transformed and/or the type of protein (e.g., antibody or fragment thereof) to be expressed. Moreover, the vector's copy number, the ability to control that copy number and the expression of any other protein encoded by the vector, such as antibiotic markers, may be considered.

In some embodiments relating to methods of producing an antibody, a host cell may be transfected with one or more expression vector(s) encoding an antibody or fragment thereof (e.g., a single vector encoding the heavy and the light chain or two vectors, one encoding the heavy chain and one encoding the light chain) and can be cultured under appropriate conditions to allow expression of the polypeptide to occur. The antibody or fragment thereof may be secreted and isolated from cells and/or cell culture media containing the antibody or fragment thereof. Alternatively, the antibody may be retained in the cytoplasm or in a membrane fraction and the harvested cells, which may be lysed subsequently purified and isolated. A cell culture includes host cells, media and other byproducts. Any suitable media for cell culture may be used in methods of production. Antibodies and antibody fragments can be isolated from cell culture medium, host cells, or both using common techniques for purifying proteins, and antibodies in particular, including, for example, ion-exchange chromatography, gel filtration chromatography, ultrafiltration, electrophoresis, and immunoaffinity purification. In certain aspects, the antibody may be produced as a fusion protein containing a domain (e.g., a His-tag) which may facilitate its purification.

A recombinant nucleic acid can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. In certain aspects, mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another aspect, the fusion gene can be synthesized by conventional techniques including automated DNA synthesizers. Alternatively, PCR amplification of gene fragments can be carried out using anchor primers which give rise to complementary overhangs between two consecutive nucleic acid fragments which can subsequently be annealed to generate a chimeric gene sequence (see, for example, Current Protocols in Molecular Biology, eds. Ausubel et al., John Wiley & Sons: 1992).

In some aspects, an expression vector expressing any of the nucleic acids described above may be used to express an antibody in a host cell. For example, an antibody may be expressed in bacterial cells such as E. coli, insect cells (e.g., using a baculovirus expression system), yeast, or mammalian cells. Other suitable host cells are known to those skilled in the art.

Once the expression vector is transferred to a host cell by conventional techniques, the transfected cells are then cultured by conventional techniques to produce an antibody. Thus, the disclosure includes host cells containing a polynucleotide encoding an antibody or antigen-binding fragments thereof, operably linked to a heterologous promoter. In certain aspects, both the heavy chain and the light chain may be co-expressed (from the same or different vectors) in the host cell for expression of the entire antibody. In certain aspects, both the heavy and light chains of the antibody are expressed from a single promoter. In certain aspects, the heavy and light chains of the antibody are expressed from multiple promoters. In certain aspects, the heavy and light chains of the antibody are encoded on a single vector. In certain aspects, the heavy and light chains of the antibody are encoded on multiple vectors.

Mammalian cell lines available as hosts for expression of recombinant antibodies are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells that possess the cellular machinery for proper processing of the primary transcript, glycosylation, and phosphorylation of the gene product may be used. Such mammalian host cells include but are not limited to CHO, HEK293, VERO, BHK, Hela, COS, MDCK, 293, 3T3, W138, BT483, Hs578T, HTB2, BT20 and T47D, NS0 (a murine myeloma cell line that does not endogenously produce any functional immunoglobulin chains), SP20, CRL7030 and HsS78Bst cells.

In certain aspects, antibodies and antibody fragments of the disclosure are stably expressed in a cell line. Stable expression can be used for long-term, high-yield production of recombinant proteins. For example, cell lines that stably express the antibody molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci, which in turn can be cloned and expanded into cell lines. Methods for producing stable cell lines with a high yield are well known in the art and reagents are generally available commercially.

In certain aspects, antibodies and antibody fragments of the disclosure are transiently expressed in a cell line. Transient transfection is a process in which the nucleic acid introduced into a cell does not integrate into the genome or chromosomal DNA of that cell, but is maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions well known in the art resulting in the expression and production of monoclonal antibodies. In certain aspects, the mammalian cell culture media is based on commercially available media formulations, including, for example, DMEM or Ham's F12. In other aspects, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium that is formulated to promote cellular growth, or cell culture production medium which is formulated to promote recombinant protein production. The terms nutrient, ingredient, and component are used interchangeably herein to refer to the constituents that make up a cell culture medium.

Once an antibody has been produced, it may be purified by any method known in the art for purification of an immunoglobulin molecule or other multimeric molecules, for example, by chromatography (e.g., ion exchange, affinity, particularly by affinity for the specific antigens Protein A or Protein G, and sizing column chromatography), centrifugation, differential solubility, or by any other standard technique for the purification of proteins.

When using recombinant techniques, the antibody and antibody fragment can be produced intracellularly, in the periplasmic space, or directly secreted into the medium. If the molecule is produced intracellularly, as a first step, the particulate debris, either host cells or lysed fragments, is removed, for example, by centrifugation or ultrafiltration. Carter et al., Bio Technology, 10:163-167 (1992) describe a procedure for isolating antibodies, which are secreted into the periplasmic space of E. coli. Where the molecule is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain, if present, in the molecule and will be understood by one of skill in the art. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the molecule to be recovered.

Regardless of how an antibody or and antibody fragments is purified, to confirm functional binding activity, binding assays may be performed (before and/or after purification). For example, ELISA assays, including dual ELISA assays may be used. In some aspects, a first antigen is coated on a well, and binding to this antigen immobilizes the antibody. A tagged second antigen is added to the well, and detected. Only antibodies that are both immobilized via binding to the first antigen and bound to the second antigen will be detected. In some aspects, the disclosure provides for recombinant cell lines that may be deposited and maintained with an international depository institution that is authorized under the provisions of the Budapest Treaty (i.e., an International Depositary Authority, IDA).

### D. Pharmaceutical Formulations

In certain aspects, the disclosure provides pharmaceutical compositions. Such pharmaceutical compositions may also be compositions comprising an antibody and/or an antibody fragment as disclosed herein and a pharmaceutically acceptable excipient. In certain aspects, the pharmaceutical compositions of the disclosure are used as a medicament (i.e., in methods of treating or preventing a disease or condition (e.g., cancer or a clinically related symptom thereof), in a subject in need of treatment or preventative treatment). In some embodiments, pharmaceutical compositions may be compositions comprising a nucleic acid molecule that encodes an antibody as disclosed herein.

In certain aspects, an antibody (or nucleic acid molecules encoding an antibody) may be formulated with a pharmaceutically acceptable carrier, excipient or stabilizer, as pharmaceutical compositions. In certain aspects, such pharmaceutical compositions are suitable for administration to a human, or a non-human mammal or animal, via any one or more route of administration using methods known in the art. The route and/or mode of administration will vary depending upon the desired results. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances, which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium and the like. These and additional known pharmaceutical carriers, excipients and/or additives suitable for use in the formulations described herein are known in the art, e.g., as listed in “Remington: The Science & Practice of Pharmacy”, 21^{st} ed., Lippincott Williams & Wilkins, (2005), and in the “Physician's Desk Reference”, 60th ed., Medical Economics, Montvale, N.J. (2005). Pharmaceutically acceptable carriers can be selected that are suitable for the mode of administration, solubility and/or stability desired or required.

The formulations described herein comprise active agents (i.e., one or more antibody or fragments thereof as disclosed herein) in a concentration resulting in a w/v appropriate for a desired dose. In certain aspects, the active agent is present in a formulation at a concentration of about 1 mg/ml to about 200 mg/ml, about 1 mg/ml to about 100 mg/ml, about 1 mg/ml to about 50 mg/ml, or about 1 mg/ml to about 25 mg/ml. In certain aspects, the concentration of the active agent in a formulation may vary from about 0.1% to about 75% by total weight. In certain aspects, the concentration of the active agent is in the range of 0.003 to 1.0 molar.

When used for in vivo administration, the formulations should be sterile. Formulations may be sterilized by various sterilization methods, including sterile filtration, radiation, etc. In one aspect, the formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005).

Thus, in embodiments, therapeutic compositions are encompassed by the pharmaceutical formulations described herein, and may formulated for particular routes of administration, such as oral, nasal, pulmonary, topical (including buccal and sublingual), rectal, vaginal and/or parenteral administration. The phrases “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The formulations may be in unit dosage form and may be prepared by any known method. Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. Suitable dosages may range from about 0.0001 to about 100 mg/kg of body weight or greater, for example about 0.1, 1, 10, or 50 mg/kg of body weight, with about 1 to about 10 mg/kg of body weight being suitable.

In some embodiments of the disclosure, the formulations may be suitable for diagnostic and research use. The concentration of active agent in such formulations, as well as the presence or absence of excipients and/or pyrogens, can be modified or selected based on the particular application and intended use.

### E. Methods

The antibodies and fragments thereof, as described herein, can be used in methods that prevent, inhibit, or reduce the activity and/or expression of GPC3 in a subject. In this aspect, the antibodies and fragments thereof disclosed herein can provide a neutralizing effect against GPC3. In some aspects, the antibodies or fragments of the disclosure can be used to treat a GPC3 condition, disease, or disorder in a subject in need of treatment. In some aspects, the antibodies and fragments disclosed herein can be used to prevent a GPC3-associated condition, disease, or disorder in a subject, who may be at risk of developing a condition, disease, or disorder. In some aspects, the antibodies can be used to treat cancer associated with GPC3 activity and/or expression, in a subject in need of treatment.

In some aspects, the disclosure relates to methods of treating, preventing, diagnosing, or monitoring, a condition, disease, or disorder characterized by GPC3, and in some embodiments the disease is a cancer. In such aspects, the antibodies, compositions and methods described herein can be used to treat a subject with a GPC3 associated condition, disease, or disorder, e.g., a cancer characterized by expression and/or activity of GPC3.

In embodiments, the disclosure provides a method of treatment comprising an antibody or antigen-binding fragment thereof in accordance with the disclosure can provide for effective treatment or “disease control” (DC). Disease control can be a complete response (CR), partial response (PR), or stable disease (SD).

A “complete response” (CR) refers to the disappearance of all tumors or lesions, whether measurable or not, and formation of no new tumor or lesions. Confirmation can be obtained using a repeat, consecutive assessment (e.g., no less than a defined number of weeks (e.g., 4) from the date of first documentation). New, non-measurable tumors or lesions preclude CR.

A “partial response” (PR) refers to a decrease in tumor burden ≥50% relative to baseline. Confirmation can be obtained using a consecutive repeat assessment (e.g., at least a defined number of weeks (e.g., 4) from the date of first documentation).

“Progressive disease” (PD) refers to an increase in tumor burden ≥25% relative to the minimum recorded (nadir). Confirmation can be obtained by a consecutive repeat assessment (e.g., at least a defined number of weeks (e.g., 4) from the date of first documentation). New, non-measurable lesions do not define PD.

“Stable disease” (SD) refers to not meeting the criteria for CR, PR, or PD. SD indicates a decrease in tumor burden of 50% relative to baseline cannot be established and a 25% increase compared to nadir cannot be established.

By “disease” is meant any condition or disorder that damages or interferes with the normal function of a cell, tissue, or organ. In the aspects and embodiments disclosed herein, the disease is typically a cancer such as, for example, a solid tumor cancer. In some embodiments, the cancer may include liver cancer. Liver cancer includes, as non-limiting examples, hepatocellular carcinoma (HCC), cholangiocarcinoma, intrahepatic cancer (IHC), or angiosarcoma. Signs associated with occurrence of liver cancer can include cirrhosis related to hepatitis B virus (HBV) and hepatitis C virus (HCV), long-term inflammation (e.g., associated with bile ducts), and increased expression or activity of GPC3, as discussed herein. While early stage liver cancer may not be associated with any symptoms or clinical indications, typical clinical symptoms associated with liver cancer (e.g., HCC and IHC) can include a lump below rib cage, pain associated with the right side of the abdomen or shoulder, jaundice, weight loss, nausea, loss of appetite, fatigue, and dark urine, among others.

Liver cancer can be diagnosed using test that are well known in the art such as, for example, blood screening to test liver function, liver enzymes, proteins (alfa-fetoprotein (AFP)) and other analytes indicative of general liver health, ultrasound, CT scan, MRI, angiogram, liver biopsy, endoscopic retrograde cholangiopancreatography (ERCP), and percutaneous transhepatic cholangiography (PTC), among others. In some embodiments in accordance with the disclosure the expression and/or activity of GPC3 can be used to assess the likelihood that a subject has or may develop a form of liver cancer.

In some embodiments of the various aspects, the disclosure relates to the determination of expression or activity of GPC3 in samples obtained from a subject who may have a cancer (e.g., solid tumor cancers, such as liver cancer), which can be used to identify patients who are likely to respond to treatment with an antibody in accordance with the disclosure. In some embodiments, the determination may comprise detecting mRNAs encoding GPC3, and can be used to identify patients who may be responsive to treatment with a GPC3 antibody. In some embodiments, the methods disclosed herein comprise detecting the amount and/or activity of GPC3 protein to determine the responsiveness of a cancer to GPC3 antibody therapy. In some further embodiments, the methods disclosed herein comprise detecting the amount and/or activity of GPC3 protein by contacting a sample (i.e., patient sample) with an antibody or antigen-binding fragment thereof in accordance with the disclosure, and detecting the presence (e.g., amount and/or activity of GPC3) in the sample. In such embodiments, the methods can be used to assess or determine the responsiveness of a cancer to GPC3 antibody therapy. The methods disclosed herein can comprise analysis of blood samples, biopsies, or immunohistochemical techniques applied to individual tissue and/or tumor samples.

In some of the above aspects and embodiments, the disclosure provides methods comprising detection of GPC3 to provide for the identification of cancers having a likelihood of positive clinical response prior to or during therapy, and also allow for modification of therapy when no signs of responsiveness, or a low likelihood of responsiveness, to a current therapy may be observable, or that may be expected based on the methods. In some embodiments, a GPC3 antibody or antigen binding fragment thereof in accordance with the disclosure can be used in the method of detection.

In one aspect, the disclosure provides a method of treating cancer in a patient comprising administering an antibody, or an antigen-binding fragment thereof as disclosed herein to the patient, wherein the patient is identified for treatment by detecting an increased level of GPC3 in a sample obtained from the patient. In some embodiments of this aspect, the disclosure provides methods for treating liver cancer in a subject.

As used herein “treat”, “treating”, or “treatment” refers to administering an antibody or antigen binding fragment thereof, or compositions as described herein to a subject in order to eliminate or reduce the clinical signs of a condition, disease, or disorder; arrest, inhibit, or slow the progression of a condition, disease, or disorder in a subject; and/or decrease the number, frequency, or severity of the clinical symptoms and/or recurrence of a condition, disease, or disorder in a subject who currently has or who previously had a condition, disease, or disorder (e.g., a cancer and/or a recurrence of cancer). In particular, the term “treatment of a disease” (e.g., disease associated with cell proliferation, cancer, etc.) includes stopping, ameliorating, reducing, shortening the duration, slowing down, or inhibiting progression or worsening, or delaying the onset of a disease or the symptoms thereof in a subject who has the disease.

In some embodiments, the disclosure provides methods for preventing disease (i.e., preventing a condition, disease, or disorder, preventing the onset of a condition, disease, or disorder or the clinical symptoms of disease) in a subject. In some further embodiments of the methods relating to preventing disease, the subject may be at risk of one or more GPC3 conditions, diseases, or disorders. By “being at risk” or having an “increased risk” a subject is identified as having a higher than normal chance of developing a condition, disease, or disorder and/or clinical symptoms associated therewith, compared to the general population. In some embodiments, a subject who has had, or who currently has, a condition, disease, or disorder, or who is of a certain age is a subject who has an increased risk for developing a condition, disease, or disorder. In some aspects, a subject exhibiting increased GPC3 expression or activity can also be considered as having an increased risk for developing a condition, disease, or disorder. In some embodiments the subject may also be immunocompromised. As used herein “immunocompromised” refers to a subject having a weakened immune system or a reduced ability to fight infections or other diseases, due to a genetic disorder or disease, an infection, an environmental disorder or disease, or other environmental factors.

The term “immunotherapy” relates to a treatment involving a specific immune reaction. In the context of the present invention, terms such as “protect”, “prevent”, “prophylactic”, “preventive”, or “protective” relate to the prevention of the occurrence and/or the propagation of a condition, disease, or disorder and clinical symptoms in an individual and, in some embodiments, to minimizing the chance that a subject will develop further clinical symptoms or disease progression. For example, a person at risk for a condition, disease, or disorder associated with GPC3, as described above, would be a candidate for preventative therapy to prevent such a condition, disease, or disorder or associated clinical symptom(s).

A prophylactic administration of an immunotherapy, for example, a prophylactic administration of an antibody or a composition comprising the antibody or fragment thereof as disclosed herein, can in certain embodiments protect the recipient from a condition, disease, or disorder associated with GPC3, or reduce the risk of the recipient from developing such a condition, disease, or disorder. In some alternative embodiments, a prophylactic administration may reduce chances of developing increased severity of a condition, disease, or disorder associated with GPC3 in the recipient.

A therapeutic administration of an immunotherapy, for example, a therapeutic administration of an antibody or a composition comprising the antibody or fragment thereof as disclosed herein, may lead to the inhibition of the progress of the condition, disease, or disorder and/or clinical symptoms associated with the condition, disease, or disorder. Such methods may include embodiments that comprise the reduction or inhibition of the expression of GPC3, the activity of GPC3, and/or the total amount of cells associated with GPC3 expression which preferably leads to elimination of the condition, disease, or disorder and the associated clinical symptoms.

In certain aspects, the disclosure provides methods of treatment comprising a GPC3 antibody or an antigen-binding fragment thereof administered in combination or in conjunction with additional cancer therapies. In particular embodiments the methods comprise a combination of GPC3 antibody or antigen-binding fragment thereof and a therapy used to treat various stages of liver cancer. Such liver cancer therapies are generally known in the art and include, without limitation, surgery (e.g., hepatectomy, liver transplant), radiofrequency ablation (i.e., microwave/thermal ablation), percutaneous ethanol injection, radiation therapy (stereotactic body radiation therapy), chemoembolization, radioembolization, and/or a targeted/systemic therapy. Non-limiting examples of a targeted or systemic therapy include, bevacizumab, atezolizumab, sorafenib, lenvatinib, cabozantinib, regorafenib, ramucirumab, pembrolizumab, nivolumab, ipilimumab, and various known combinations thereof.

The terms “subject”, “individual” or “patient” are interchangeable, and relate to vertebrates, preferably mammals. For example, mammals in the context of the disclosure are humans, non-human primates, domesticated animals such as dogs, cats, sheep, cattle, goats, pigs, horses etc., laboratory animals such as mice, rats, rabbits, guinea pigs, etc., as well as animals in captivity such as animals in zoos. The term “animal” as used herein also includes humans. The term “subject” may also include a patient, i.e., an animal, an in particular embodiments a human having a disease associated with GPC3. Subjects may also include a patient who may be at risk of developing a condition, disease, or disorder associated with GPC3. In particular embodiments the subject, individual, or patient is a human.

The methods comprising the antibodies or binding fragments thereof as described herein, and compositions comprising the same, may be administered via any conventional route, including by injection or infusion, oral, buccal, sublingual, transdermal, intraocularly, intranasally, by aerosol, by implant or depot, or intrarectally. In some embodiments, the administration may be carried out, for example, by injection such as, intravenously, intraperitonealy, intramuscularly, subcutaneously, or transdermally.

The antibodies, fragments thereof, and the compositions comprising them are administered in effective amounts. An “effective amount” includes an amount that achieves a desired reaction or a desired effect and may be in the form of a single dose or as multiple doses. In the case of treatment of a particular disease or of a particular clinical condition, the desired reaction relates to inhibition of the course of the disease or symptom. Such inhibition can include slowing down the progression of the disease/clinical symptoms and, in some embodiments, interrupting or reversing the progression of the disease/clinical symptoms. The treatment of a disease or of a condition may also be delay of the onset or a prevention of the onset of said disease or said condition in a subject who has active disease such as, for example, a cancer.

An effective amount of a composition of the invention will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses of the compositions of the invention administered may depend on various combinations of such parameters. In embodiments in which an initial amount administered to a patient is insufficient, further administration with higher amounts, more frequent doses, or a different/more localized route of administration may be used.

In accordance with the above aspects, in some embodiments the disclosure relates to methods of monitoring a condition, disease, or disorder associated with a GPC3 expression in a subject, wherein the methods comprise the detection and/or the determination or the monitoring of the quantity of (i) a GPC3 nucleic acid, (ii) a GPC3 antigen or a part thereof, (iii) an antibody against GPC3 or a part thereof, and/or (iv) a cell comprising GPC3, in a biologic sample isolated from a patient. In some further embodiments, the condition, disease, or disorder associated with GPC3 expression comprises a cancer.

In accordance with the above aspects, in some embodiments the disclosure relates to a method of diagnosing a condition, disease, or disorder characterized by GPC3 expression. In some embodiments, the method comprises the detection, and/or the determination of the quantity, of (i) a GPC3 nucleic acid, (ii) a GPC3 antigen or a part thereof, (iii) an antibody against GPC3 or a part thereof, and/or (iv) a cell comprising GPC3, in a biologic sample isolated from a patient. In some further embodiments, the condition, disease, or disorder associated with GPC3 expression comprises a cancer.

In some embodiments of the above aspects, detection comprises (i) contacting the biological sample with an antibody or antigen-binding fragment thereof as disclosed herein, which binds specifically to GPC3 or a part thereof, an antibody against GPC3 or a part thereof, and/or a cell comprising surface expression of at least a portion of GPC3 antigen, and (ii) detecting the formation of a complex between the antibody or antigen-binding fragment thereof and GPC3 or the part thereof, the antibody against GPC3 or a part thereof, and/or a cell comprising a GPC3 antigen. In some embodiments, the biological sample isolated from the patient is compared to a reference sample, e.g., a comparable biological sample obtained from a healthy subject having normal levels of GPC3 expression.

In a further aspect, the disclosure relates to a method for determining regression, course, or onset of a disease characterized by GPC3 expression. In some embodiments the disease may be identified according to various methods provided by the disclosure. In some embodiments, the method comprises monitoring a sample from a patient who has said disease or is suspected of having said disease, for one or more parameters selected from the group consisting of (i) the amount of nucleic acid associated with GPC3, (ii) the amount of expressed GPC3 antigen or a part thereof, (iii) the amount of an antibody against GPC3 antigen or a part thereof, and/or (iv) the amount of a cell associated with GPC3 expression. In some, embodiments the method comprises determining the parameter(s) in a first sample at a first point in time and in a further sample at a second point in time and in which the course of the disease is determined by comparing the two samples.

The term “sample” can include any sample that is useful or usable in accordance with the methods described herein. In some embodiments a sample can be a biological sample, such as a sample from a tissue, which may include, for example, bodily fluids, and/or cellular samples that may be obtained by any conventional method (e.g., by tumor/tissue biopsy, taking blood, bronchial aspirate, sputum, urine, feces or other body fluids). In some embodiments, the term “sample” may include processed samples such as fractions or isolates of biological samples, e.g. nucleic acid and peptide/protein isolates.

According to some embodiments, detection of a GPC3 antigen, or associated cell expressing a GPC3 antigen or of a fragment thereof, or determining or monitoring the amount of a GPC3 antigen or cell associated with at least a portion of a GPC3 antigen may be carried out using one or more antibodies, constructs, or fragments as described herein. The methods comprising detection that incorporate an antibody or fragment thereof as disclosed herein may comprise a detectable label. In some embodiments, the detectable label or marker is a fluorescent, colorimetric, radioactive, or an enzymatic marker, or other markers as disclosed herein or otherwise known in the art.

As discussed above, the methods provided herein can decrease, slow, or halt tumor growth and, in some aspects the reduction or slowing may be statistically significant. A reduction in tumor growth can be measured by comparison to the growth of patient's tumor at baseline, against an expected tumor growth, against an expected tumor growth based on a large patient population, or against the tumor growth of a control population.

In certain aspects, administration of a GPC3 antibody or an antigen-binding fragment thereof can increase overall survival (OS). In other aspects, administration of a GPC3 antibody or an antigen-binding fragment thereof can provide stable disease (SD).

### F. Kits

Another aspect of the present disclosure is a kit. In one aspect, a kit comprises any of the sequences, compounds and pharmaceutical formulations or compositions of a nucleic acid, polypeptide, expression vector, or host cell as generally described above, and instructions or a label directing appropriate use or administration. Optionally, a kit may also include one or more containers reagents, reactants, and/or a syringe or other device to facilitate delivery or use. The disclosure contemplates that all or any subset of the components for conducting research assays, diagnostic assays and/or for administering therapeutically effective amounts may be enclosed in the kit. Similarly, the kit may include instructions for making an amino acid sequence/polypeptide by, for example culturing a host cell that expresses a nucleic acid that encodes an antibody (or construct or antigen binding fragment thereof) of the disclosure under suitable conditions. By way of additional example, a kit for therapeutic administration of an antibody of the disclosure may comprise a solution containing a pharmaceutical formulation of the antibody, or a lyophilized preparation of the antibody, and instructions for administering the composition to a patient in need thereof and/or for reconstituting the lyophilized product.

The present disclosure also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient, e.g., an above-described antibody, is sterile and suitable for administration as a particulate free solution. In certain aspects, the formulation is suitable for intravenous administration, such as for intravenous infusion to a human or animal.

In a specific aspect, the formulations of the disclosure are formulated in single dose vials as a sterile liquid. Exemplary containers include, but are not limited to, vials, bottles, pre-filled syringes, IV bags, and blister packs (comprising one or more pills). Optionally associated with such container(s) can be a notice in the form prescribed by a governmental agency regulating the manufacture, use or sale of pharmaceuticals or biological products, which notice reflects approval by the agency of manufacture, use or sale for human diagnosis and/or administration.

As with any pharmaceutical product, the packaging material and container are designed to protect the stability of the product during storage and shipment. Further, the products of the disclosure include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, etc., and other monitoring information.

A kit for diagnostic assays may comprise a solution containing an antibody, or antigen-binding fragment thereof, or a lyophilized preparation of an antibody or fragment thereof of the disclosure, wherein the antibody or fragment binds specifically to one or more targets, as well as reagents for detecting such antibodies. The antibody may be labeled according to methods known in the art and described herein, including but not limited to labels such as small molecule fluorescent tags, proteins such as biotin, GFP or other fluorescent proteins, or epitope sequences such as his or myc. Similarly, primary antibodies used for detecting the antibody may be included in the kit. Primary antibodies may be directed to sequences on the antibody or to labels, tags, or epitopes with which the antibodies are labeled. Primary antibodies may, in turn, be labeled for detection, or, if further amplification of the signal is desired, the primary antibodies may be detected by secondary antibodies, which may also be included in the kit.

Kits for research use are also contemplated. Such kits may, for example, resemble kits intended for diagnostic or therapeutic uses but further include a label specifying that the kit and its use is restricted to research purposes only.

G. Modification and/or Engineering

Another aspect of the disclosure includes the modification and/or engineering of the antibodies and/or antigen binding fragments thereof that are described herein. For example, antibodies and/or antigen binding fragments thereof include, without limitation, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual-specific antibody, and a bispecific antibody. Non-limiting embodiments of such constructs and antibody formats are illustrated in the Examples and throughout the disclosure. The disclosed antibodies, and antigen binding fragments thereof, can also be used to generate fusion proteins and/or for antibody-targeted cell fusion, such as with immunological fusion partners and cells.

The disclosed antibodies, and antigen binding fragments thereof, can also be used to produce therapeutic immunoconjugates, wherein the disclosed antibodies, or antigen binding fragments thereof, are conjugated with one or more therapeutic agents. Non-limiting embodiments of such immunoconjugates are illustrated throughout the disclosure. In addition, for example, disclosed antibodies, or antigen binding fragments thereof, can be used in the production of Antibody-Drug-Conjugates (ADC). Drugs that can be used include, without limitation, inhibitors of cell proliferation, anticancer agents, agents that induce apoptosis, and the like, and combinations thereof.

As discussed herein, the practice of the methods disclosed herein employs, unless otherwise indicated, available techniques of molecular biology (including recombinant techniques), microbiology, cell biology, biochemistry and immunology, which are familiar to the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991).

The following examples provide an illustration of some of the aspects and embodiments described above, and are not intended to limit the scope of the claimed invention.

### EXAMPLES

### Antibody Generation, Phage Display, and Affinity Maturation

Antibody generation was accomplished using the Glypican 3 Protein (GPC3) as the panning antigen (Sino Biological, Cat #10088-H08H-B). Fully human scFv libraries containing either IgM (1×10⁵-2×10⁹ colonies per round) or IgG (1×10⁵-2×10⁸ colonies per round) were constructed and panned against the GPC3 antigen. After three rounds of panning, clones that were positive in phage ELISA were sequenced. More than 40 unique sequences were obtained from over 200 sequenced clones. They were converted to scFv-Fc for expression and functional screenings. The examples below provide details for 8 representative clones that were characterized in-depth. One of the 8 representative clone sequences was used to develop a series of affinity matured constructs (using phage display) that focused on modifications to the VH region to screen for and assess improved GPC3 binding affinity.

Nine affinity-matured clones were selected for further characterization. The resulting data detailed below demonstrate that the disclosure provides for antibodies (i.e., GPC3 binding antibody constructs/fragments) having excellent specific binding properties to GPC3 and provide for their use, alone or as immunoconjugates, in a variety of applications such as, for example, methods of treatment (e.g., methods that inhibit/neutralize GPC3 biological activity, methods for treating cancer) as well as for use in GPC3 assays and diagnostic applications.

### Example 1—Generating and Screening IgGs that Bind GPC3 Ligand

Direct ELISA assay is the primary assay used to define the binding specificity of the mAbs identified from the fully human scFv phage libraries.

From the scFv libraries, 16 candidates were selected for scFv-Fc format expression in mammalian expression host cells, and were isolated and purification for functional analysis. Heavy chain variable (VH) and light chain variable (VL) sequences were fused to human IgG1 constant regions and transfected into 293F cells transiently.

For ELISA analyses, Glypican 3 Protein, Human, Recombinant (His Tag) (Sino Biological, Cat #10088-H08H) was coated on a 96-well ELISA plate (Thermo Cat #3855, Immulon 4 HBX) at 1 μg/ml in Phosphate Buffered Saline (PBS) at 4° C. overnight. The plate was then incubated with supernatant containing scFv-Fc expressed from HEK293 cells with serial dilutions in PBST buffer (PBS+0.1% Tween)+3% dry milk. The secondary antibody, goat anti-human Fc-HRP (Invitrogen, Cat #A18829), was added at 0.5 ug/ml final concentration in PBST+3% dry milk. All steps were incubated for 1 hour at room temperature. Plates were washed three times with PBST between each incubation step. After incubation with the secondary antibody, HRP substrate 1-step Ultra TMB-ELISA (Thermo, Cat #34029) was added to develop the colorimetric signal. The HRP reactions were stopped by addition of 2 M sulfuric acid. Measurements were made (OD₄₅₀) on a SpectraMax M3 plate reader (Molecular Devices, CA, USA). ELISA binding results were ranked, and top binders were selected for further analyses.

Table 1 details sequence information for several illustrative GPC3 binding antibody constructs in accordance with the example embodiments as disclosed herein.

TABLE 1
Heavy Chain and Light Chain Variable Region Sequences for Representative GPC3
Antibodies, with identified CDR regions (bolded, underlined)

| Ref | Variable | |
|---|---|---|
| ID | Chain | Sequence |
| P1 | Heavy | QVQLQQSGPGLVKPSQTLSLTCAISGDSVSSYSAAWNWIRQSPSRGLEWL |
| | | GRTYYRSKWYNDYEASVKSRITFKADTSKNQLSLQLNSVTPEDTAVYYC |
| | | ARGQWRFDYWGQGTLVTVSS |
| | | (SEQ ID NO: 1) |
| | Light | SYELTQPPSVAVAPGQTARITCEEYNIESKSVHWYQEKPGQAPVLVIYND |
| | | GDRPSGVPERFSGSNSGNTATLTINRVEAGDEAEYYCXAWDIGTDHPIFG |
| | | VGTKLTVL |
| | | (SEQ ID NO: 2) |
| P3 | Heavy | QVQLQQSGPGLVKPSQTLSITCAISGDSVSSNSAAWNWIRQSPSRGLEWL |
| | | GRTYYRSKWYNDYAVSVKSRITINPDTSKNQFSLQLNSVTPEDTAVYYC |
| | | ASSYYNGGYFDYWGQGTLVTVSS |
| | | (SEQ ID NO: 3) |
| | Light | EIVLTQSPATLSVSPGGRATLSCRASQSVTTNLAWYQQKPGQAPRLLIYG |
| | | ASTRVTGVPRRFSGSGSGTDFTLTISSLQSEDSAVYYCQQYSGWGGFGG |
| | | GTKVDIK |
| | | (SEQ ID NO: 4) |
| P10 | Heavy | EVQLVQSGAEVKKPGSSVKVSCKASGGTFRSYAISWVRQAPGQGLEWM |
| | | GGIIPIFGTANYAQKFQGRVTITADELTSTAYMELSSLRSEDTAVYYCAS |
| | | RIGQWPTPFDYWGQGTLVTVSS |
| | | (SEQ ID NO: 5) |
| | Light | QSVLTQPPSVSVAPGKTARITCGGNNIGSKSVHWYQQKPGQAPVLVIYY |
| | | DSDRPSGIPERFSGSNSGNTATLTISRVEAGDEADYYCQVWDSSSDHPVF |
| | | GGGTKLTVL |
| | | (SEQ ID NO: 6) |
| P17 | Heavy | EVQLVESGGGLVQPGGSLRLSCAASGFTFRSYAMSWVRQAPGKGLEWV |
| | | SAISGSGGRTYYADSVKGRFTISRDNSKNTLYLQMNSLRAEDTAVYYCA |
| | | KEAPYSSGWSHYYYGMDVWGQGTTVTVSS |
| | | (SEQ ID NO: 7) |
| | Light | QSVLTQPPSVSGAPGQRVTISCTGSSSGYDVHWYQQLPGRAPKLLIYGSN |
| | | NRPSGVPDRFSGSNSGTSASLAITGLQAEDEADYYCQSYDSSLNAIFGGG |
| | | TKLTVL |
| | | (SEQ ID NO: 8) |
| P19 | Heavy | EVQLVESGGGLVKPGGSLRLSCAASGFTFSDYYMSWIRQAPGKGLEWVS |
| | | YISSSGSTIYYADSVKGRFTISRDNAKNSLYLQMNSLRAEDTAVYYCASV |
| | | YMDVWGKGTTVTVSS |
| | | (SEQ ID NO: 9) |
| | Light | EIVMTQSPGTLSLSPGERATLSCRASQTVSSSSLAWYQQRPGQAPRLLIYE |
| | | TSSRATGIPDRFSGSGSGTDFTLTISRLEPEDFAIYYCQQKGDSGYTFGQG |
| | | TKVEIK |
| | | (SEQ ID NO: 10) |
| P20 | Heavy | EVQLVESGGGLVKPGGSLRLSCAASGFTFSDYYMSWIRQAPGKGLEWVS |
| | | YISSSGSTIYYADSVKGRFTISRDNAKNSLYLQMNSLRAEDTAVYYCASV |
| | | YMDVWGKGTTVTVSS |
| | | (SEQ ID NO: 11) |
| | Light | SYELTQPPSVSVAPGQTARITCGGNNIGSKSVHWYQQKPGQAPVLVVYD |
| | | DSDRPSGVPERFSGSNSGNTATLTISRVEAGDEADYYCQVWDNSSDHQV |
| | | FGGGTKLTVL |
| | | (SEQ ID NO: 12) |
| 59 | Heavy | QVQLVESGAEVKKPGASVKVSCKASGYTFTGYYMHWVRQAPGQGLEW |
| | | MGRINPNSGGTNYAQKFQGRVTMTRDTSISTAYMELSRLRSDDTAVYYC |
| | | ARASGRVTDAFDIWGQGTMVTVSS |
| | | (SEQ ID NO: 13) |
| | Light | QAVLTQPSSLSASPGASASLTCTLRSGINVVPYRIYWYQQKPGSPPQFLLN |
| | | YRSDSDNYQGSGVPSRFSGSKDTSANAGILLISGLQSEDEADYYCLTWHS |
| | | DSWVFGGGTKLTVL |
| | | (SEQ ID NO: 14) |
| 71 | Heavy | QVQLVESGGGVVQPGRSLRLSCAASGFTFSSYAMHWVRQAPGKGLEWV |
| | | AVISYDGSNKYYADSVKGRFTISRDNSKNTLYLQMNSLRAEDTAVYYCA |
| | | RGGDGYNAAFDIWGQGTMVTVSS |
| | | (SEQ ID NO: 15) |
| | Light | NFMLTQPPSVSESPGKTVTISCTGSSGSIASNYVQWYQQRPGSAPTTVIYE |
| | | DNQRPSGVPDRFSGSIDSSSNSASLTISGLKTEDEADYYCQSYDSSKHYVF |
| | | GTGTKVTVL |
| | | (SEQ ID NO: 16) |

### Example 2—GPC3 Binding

Binding affinity was measured using a three-step protocol using Octet QKe (ForteBio). In the first step, seven Anti-hIgG Fc Capture (AHC) biosensor probes were loaded by contacting them with seven different antibody solutions (2.5 μg/ml, in separate microplate wells) of the testing antibody. A reference probe was loaded with the same amount of a control antibody. In the second step, the loaded biosensors probes were contacted with antigen using wells containing antigen in a series of concentrations, 0 nM, 5.625 nM, 11.25 nM, 22.5 nM, 45 nM, 90 nM, 180 nM, and 360 nM. A separate single antigen well (360 nM) was set up for the reference probe. The reactions were monitored for 300 seconds. In the third step, the probes were transferred into buffer solutions to initiate the dissociation reactions. The dissociation reactions were monitored for 600 seconds. After measurement, the probes were regenerated according to the manufacturer's instructions. The resulting data were fit to a 1:1 binding model using Octet data analysis software (see, Table 2).

TABLE 2
K_{D} measurement of selected candidates by Octet

| Candidate | Ligand | K_{D} | |
|---|---|---|---|
| P1 | Hu-GPC3 (10088-H08H) | 4.76 | nM |
| P1 | Cy-GPC3 (GP3-C5225) | 1.84 | nM |
| P3 | Hu-GPC3 (10088-H08H) | 20.1 | nM |
| P3 | Cy-GPC3 (GP3-C5225) | 5.45 | nM |
| P10 | Hu-GPC3 (10088-H08H) | 2.85 | nM |
| P10 | Cy-GPC3 (GP3-C5225) | 1.28 | nM |
| P17 | Hu-GPC3 (10088-H08H) | 9.1 | nM |
| P17 | Cy-GPC3 (GP3-C5225) | 6.01 | nM |
| P20 | Hu-GPC3 (10088-H08H) | 100 | nM |
| 59 | Hu-GPC3 (10088-H08H) | 11.3 | nM |
| 59 | Cy-GPC3 (GP3-C5225) | 6.49 | nM |

### Example 3—Epitope Binning

A sandwich-style epitope binning was used to detect whether the illustrative antibodies can function to block one another's binding to an epitope of the GPC3 antigen. Briefly, a first anti-GPC3 scFv-Fc was diluted to 10 nM in buffer and was captured by an AHC biosensor. The biosensor was then transferred to the antigen well containing 300 nM human Glypican 3 protein (Sino Biological, Cat #10088-H08H) in the same buffer to initiate the antigen binding (phase I association). Following the initial binding, the biosensor was dipped into another well containing 200 nM of a second anti-GPC3 scFv-Fc to start the phase II association. A slower association rate in phase II compared to that in phase I is indicative of competition at the epitope (e.g., blocking binding). Antibodies that block binding to the same epitope are binned together. The results indicate that P17, P19, and P20 share a substantially similar epitope, P1 and P10 share a common but different epitope, and each of P3, 59, and 71 appear to bind to a unique epitope (Table 3).

TABLE 3
GPC3 scFv-Fc binning data

| Binning group | Construct |
|---|---|
| 1 | P17, P19, P20 |
| 2 | P3 |
| 3 | P1, P10 |
| 4 | 59 |
| 5 | 71 |

### Example 4—Cell Surface Binding

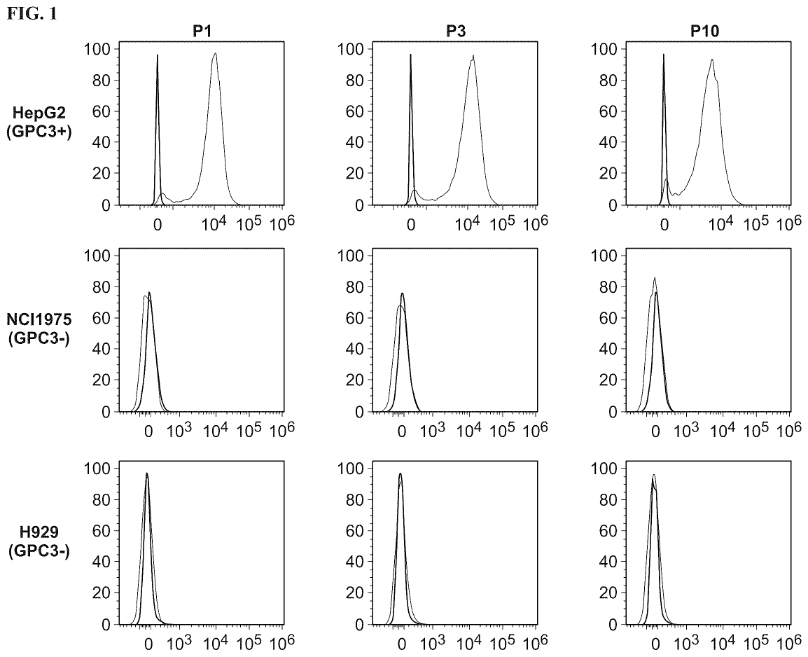

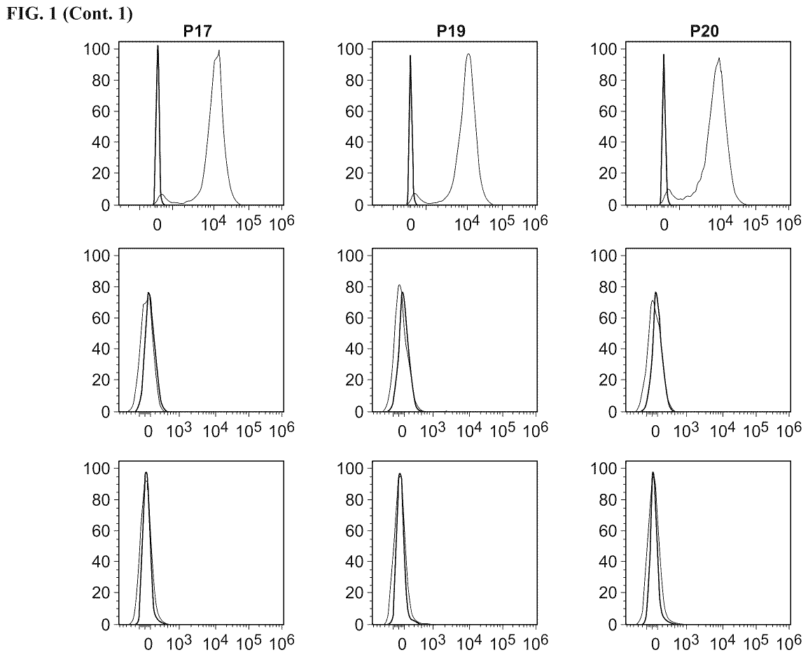

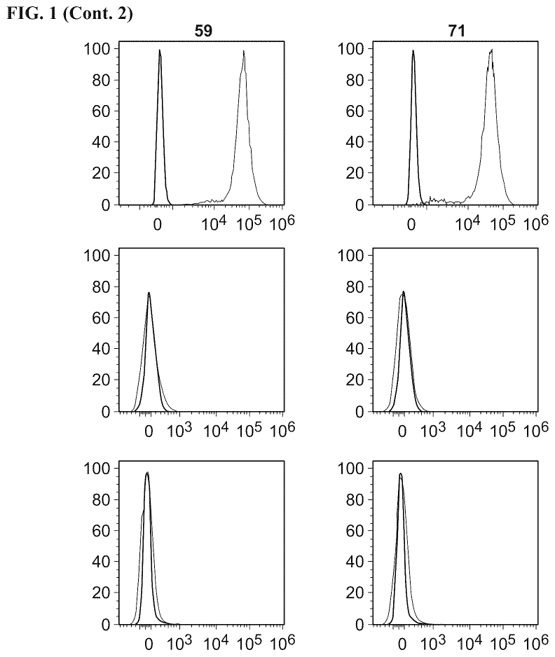

Glypican 3 protein is expressed on the surface of mammalian cells which allows for cell-based assays to assess and analyze binding values. GPC3 positive HepG2 cells and GPC3 negative NCI-H1975 and NCI-H929 cells were used for cell surface binding measurements. Cells were plated at 1×10⁵ to 5×10⁵ cells per well in a 96-well plate and incubated on ice for 45 min with anti-GPC3 scFv Fc fusion proteins. The cells were washed twice, followed by addition (at 1:1000) of goat anti-Human IgG (H+L) conjugated with Alexa Fluor™ 647 (Invitrogen, cat #A21445) to detect the binding scFv-Fc. Analysis of cell surface-bound fluorescence was measured using Attune NxT flow cytometry (Thermo Fisher Scientific). A shift of fluorescent signals in the GPC3 positive HepG2 cells confirmed the cell surface binding of candidates P1, P3, P10, P17, P19, P20, 59, and 71, while the lack of shift in the GPC3 negative NCI-H1975 and NCI-H929 cells indicated the ligand specificity of these candidates (see, FIG. 1).

### Example 5—Cell Internalization

Ligand internalization via receptor-mediated endocytosis is useful in determining whether an extracellular molecule binds to its specific receptor protein at the cell surface. To evaluate the ligand internalization of the illustrative GPC-antibody constructs into GPC3 expressing cells, the scFvs were converted to Fab format and incubated with HepG2 and Huh7 cells. Fabs were diluted to 100 μg/mL with cold FACS buffer. In a 96-well plate each of P3, P10, P17, P20, 59, and 71 Fabs, (100 μL), or control (100 μL FACS buffer) was added to separate wells containing HepG2 and Huh7 cells, and incubated on ice for 30 minutes, allowing for antibody (Fab) binding to all antigen sites on the target cells.

Except for a control samples (t=0 h), the cells were incubated at 37° C. to induce endocytosis of bound proteins. Samples were harvested at various time points (0 h, 1 h, 2 h, 3h, and 4 h) and were immediately washed twice with cold FACS buffer to remove any unbound Fab. The samples were fixed with 4% paraformaldehyde at room temperature for 20 min. After all the samples were collected, 1 μl FITC fluorescent-labeled anti-human IgG (H+L) secondary antibody was added to each sample and incubated at 4° C. for 30 min. The cells were washed twice with FACS buffer, and resuspended in 100 μL FACS buffer for analysis using a CytoFLEX flow cytometer (Beckman Coulter). The presence of internalized Fab (int. Fab) was indicated by a decrease in median fluorescence intensity (MFI) using the formula:

MFI of int. Fab=MFI of the sample at 0h−MFI of the sample at different time points.

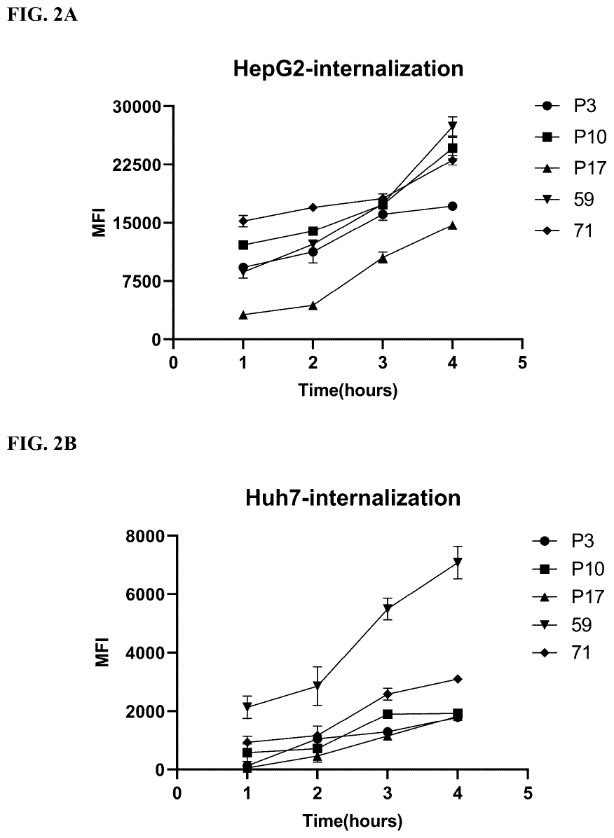

As shown in FIG. 2, the amount of internalized Fabs increased over time, and different internalization rates were observed between the different cells (HepG2 (top panel) and Huh7 (bottom panel)). The higher MFI observed for HepG2 indicated that higher amounts of GPC3 ligand expressed on the surface of HepG2 cells, relative to Huh7 cells. The increase in MFI over time indicated more internalization in both HepG2 and Huh7 cells. While various factors may contribute to this observation, the higher average internalization rate typically associated with HepG2 cells relative to Huh7 cells can correlate to a higher level of expression of GPC3 in HepG2 cells.

### Example 6—Cancer Cell Inhibition

Immunoconjugates of GPC3 binding molecules, constructed as Fabs and conjugated to a fragment of Pseudomonas exotoxin A were recombinantly prepared as candidate immunotoxin (RIT) molecules. The RITs bind to GPC3 at the cell surface via the Fab fragment, and induce cell apoptosis by inhibiting protein synthesis in target cells upon internalization. The inhibition of cell proliferation and cytotoxicity of P10, P17, 59, 71 RIT constructs against HepG2 and Huh7 cell lines was evaluated by CCK8 assay.

Briefly HepG2 and Huh7 cells were seeded at 5000 cells per well on 96-well plates for 24 h. After attachment, different concentrations of P10, P17, 59, 71 RITs (25, 1250, 5000, 15000, 25000, 40000, 50000, 100000, 200000, 400000 ng/ml) were added to the cells in triplicate and incubated for 72 h under standard culture conditions. After 72 h, the medium was removed and 10 μL of CCK8 solution with 90 μL fresh medium was added to each well. Plates were incubated for 0.5-1 h at 37° C. The absorbance of each well was detected at 450 nm wavelength by a microplate reader (Tecan), and the percentage of cell viability was calculated by the following formula: (viable cells) %=(OD of immunotoxin-blank sample/OD of control-blank sample)×100. The IC₅₀ values of immunotoxins were analyzed by non-linear regression analysis using a fitting formula of log (inhibitor) vs. response (three parameters) (GraphPad Prism 8.0.2, San Diego, California).

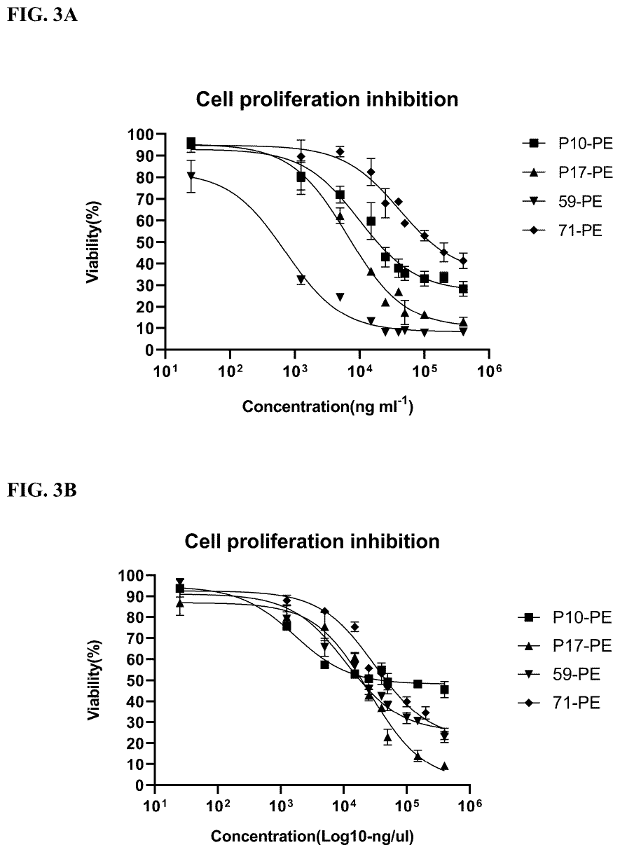

As shown in FIGS. 3A-3B, the GPC3 RITs exerted an inhibitory effect on the proliferation of the HepG2 (FIG. 3A) and Huh7 (FIG. 3B) cell lines in a dose-dependent manner. Table 4 summarizes the two RITs with the lowest IC50s measured. The data indicate the successful internalization of RITs constructed with GPC3 binding sequences and their utility as therapeutics for conditions associated with uncontrolled cell proliferation (e.g., cancer). Different RITs constructed with different lead candidate sequences of varying binding affinities resulted in different observed cytotoxicities.

TABLE 4
Immunoconjugate inhibition

| | |
|---|---|
| IC₅₀ against HepG2 | IC₅₀ against Huh7 |

| | | | | |
|---|---|---|---|---|
| P17 RIT | 59.9 | nM | 231.3 | nM |
| 59 RIT | 6.5 | nM | 99.36 | nM |

### Example 7—Immunoconjugate Induced Cell Apoptosis

The ability of GPC3 RITs to induce apoptosis/necrosis in HepG2 cells was analyzed using Annexin V and PI staining. Briefly, HepG2 cells were seeded overnight into 12-well plates (2×10⁵ cells/well). After attachment, RITs (P3, P10, P17, 59, 71) were diluted with cell culture medium to concentrations of 5000, 10000, 15000, 30000 ng/ml, added to the wells and incubated for 24 h. After incubation, cells were collected by trypsin (without EDTA) digestion and washed with precooled PBS twice and resuspended with precooled 1× binding buffer (Annexin V-FITC/PI apoptosis kit, Multi sciences). The blank/control group was divided into two tubes; the first tube was incubated with 500 μL apoptosis positive control solution, and the second tube incubated in precooled PBS for 30 min on ice. After washing twice with precooled PBS, 1 μL Annexin V-FITC or 2 μl PI was added to the control and experimental groups, gently mixed, and incubated at room temperature for 5 min protected from light. The samples were analyzed by CytoFLEX flow cytometer (Beckman Coulter). The proportion of apoptotic cells was calculated as follows: % Apoptosis=(% Annexin-V-positive cells+% double positive cells)/total cells×100.

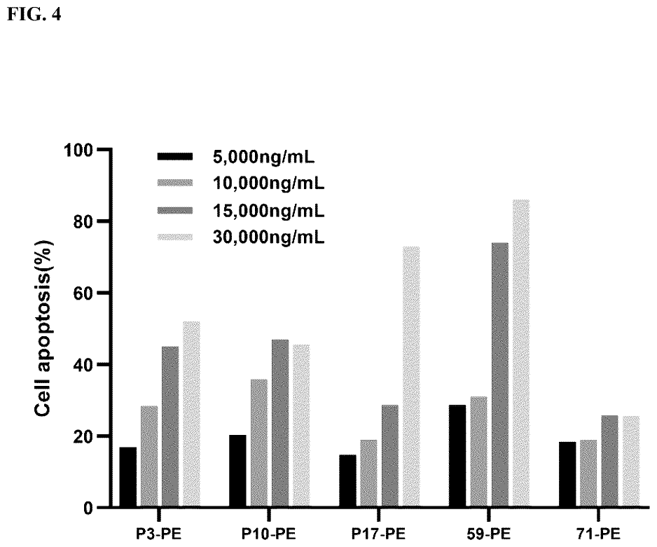

As shown in FIG. 4, the GPC3 RITs exerted an apoptotic effect on the HepG2 cells in a dose-dependent manner, indicating the successful internalization of RITs constructed with GPC3 binding sequences and validating a mechanism of action useful in cancer therapy. Different RITs constructed with different lead sequences possessing varying binding affinities resulted in different cytotoxicity.

### Example 8—T cell Activation of GPC3-CD3 Bispecific Antibody Constructs

A series of bispecific antibodies were constructed using the GPC3 antibody sequences described herein and a CD3 antibody scFv. The bispecific antibodies were evaluated for their effects in bridging cells expressing GPC3 and activating CD3 positive T cells. Briefly, a CD3 scFv was engineered onto the C terminus of a GPC3 mAb light chain and connected by a linker sequence. The bispecific antibody was expressed in 293F cells and was recovered and purified using a MabSelect SuRe column (Cytiva, Cat #28926977). Target cells expressing GPC3 (e.g. HepG2 at 25,000 cells per well) and effector cells (e.g. Jurkat at 120,000 cells per well) were seeded in a 96-well plate. Purified bispecific antibodies were added to the cell culture in a range of concentrations and incubated for 7 h before an endpoint reading was taken for T cell activation.

Wells seeded with the effector cells but without the GPC3 target cells were used as a negative control. Bispecific antibodies constructed with variable domains from P1, P3, P10, P17, and 59, when combined with a CD3 scFv successfully induced strong activation of T cells when GPC3 positive target cells were present, and no or minimal activation in the presence of GPC3 negative NCI-H1975 cells, or in the absence of target cells, as summarized in Table 5.

TABLE 5
T cell activation efficacies of GPC3-CD3 bispecific antibodies

| | P1 | | P3 | | P10 | | P17 | | 59 | |
|---|---|---|---|---|---|---|---|---|---|---|
| EC₅₀ against CHOK1/GPC3 (++) | 74 | pM | 171 | pM | 91 | pM | 63 | pM | 63 | pM |
| EC₅₀ against HepG2 (+) | 137 | pM | 182 | pM | 85 | pM | 40 | pM | 68 | pM |
| EC₅₀ against Hep3B (+) | 102 | pM | 314 | pM | 40 | pM | 27 | pM | 49 | pM |
| EC₅₀ against NCI-H1975 (−) | >20 | nM | >64 | nM | >12 | nM | >50 | nM | >79 | nM |

### Example 9—Affinity Maturation

Random mutations were introduced into the CDRs described above in the heavy and light chain variable region sequences (see Table 1) using degenerate oligos, resulting in a total of 11 libraries. The design of the oligos is summarized in Table 6. Libraries constructed to cover the same CDR were combined for panning. Two rounds of panning were performed with libraries covering each CDR, except for those libraries covering LCDR3. Briefly, biotinylated GPC3 (Sino Biological, cat #10088-H08H-B) was added to M-280 Streptavidin Dynabeads™ (Invitrogen, cat #11205D) and then incubated with the phage library. Unbound and weakly-bound phages were washed away with PBST (0.1% Tween-20 in PBS). Phages displaying relatively higher affinity scFvs remained on the beads and were eluted with 0.1 N HCl and rescued with log-phase TG1 cells. Single colonies of these cells were picked randomly and sent for sequencing. After one round of panning, most of the sequenced colonies from LCDR3 libraries had the same sequence as the “WT”, indicating that the LCDR3 sequence may play a key role (immutable) in the GPC3 antibody-antigen interaction. For that reason, the LCDR3 sequence was held constant.

TABLE 6
Oligonucleotide design

| | | Target | | |
|---|---|---|---|---|
| CDR | Library | sequence | Forward primer (5′→3′) | Reverse primer (5′→3′) |
| HCDR1 | HC1.1 | GFTFR | GCCTCTNHSNHSNHSNHSNHS | TGCACAGGAGAGTCTCAGGG |
| | | (SEQ ID: | AGCTATGCCATGAGCTGGGT | AC |
| | | 85) | C | (SEQ ID NO. 18) |
| | | | (SEQ ID NO. 17) | |
| | HC1.2 | FRSYA | TTCACCNHSNHSNHSNHSNHS | TCCAGAGGCTGCACAGGAG |
| | | (SEQ ID: | ATGAGCTGGGTCCGCCAG | (SEQ ID NO. 20) |
| | | 86) | (SEQ ID NO. 19) | |
| HCDR2 | HC2.1 | ISGSG | TCAGCTNHSNHSNHSNHSNHS | GACCCACTCCAGCCCCTTCC |
| | | (SEQ ID: | GGTAGAACATACTACGCAGA | (SEQ ID NO. 22) |
| | | (87) | CTCCGTG | |
| | | | (SEQ ID NO. 21) | |
| | HC2.2 | SGGRT | AGTGGTNHSNHSNHSNHSNHS | AATAGCTGAGACCCACTCCA |
| | | (SEQ ID: | TACTACGCAGACTCCGTGAA | GC |
| | | 88) | GGG | (SEQ ID NO. 24) |
| | | | (SEQ ID NO. 23) | |
| HCDR3 | HC3.1 | EAPYS | GCGAAANHSNHSNHSNHSNH | ACAGTAATATACGGCCGTGT |
| | | (SEQ ID: | SAGTGGCTGGTCTCACTACTA | CCTCG |
| | | 89) | CTACG | (SEQ ID NO. 92) |
| | | | (SEQ ID NO. 25) | |
| | HC3.2 | SGWSH | TATAGCNHSNHSNHSNHSNHS | CGGGGCTTCTTTCGCACAG |
| | | (SEQ ID: | TACTACTACGGTATGGACGTC | (SEQ ID NO. 27) |
| | | 90) | TGGGG (SEQ ID NO. 26) | |
| | HC3.3 | HYYYG | TGGTCTNHSNHSNHSNHSNHS | GCCACTGCTATACGGGGCTTC |
| | | (SEQ ID: | ATGGACGTCTGGGGCCAAG | (SEQ ID NO. 29) |
| | | 91) | (SEQ ID NO. 28) | |
| LCDR1 | LC1.1 | SSGYD | GGGAGCNHSNHSNHSNHSNH | AGTGCAGGAGATGGTGACCC |
| | | (SEQ ID: | SGTACACTGGTACCAGCAGCT | TC |
| | | 93) | TCC | (SEQ ID NO. 31) |
| | | | (SEQ ID NO. 30) | |
| LCDR2 | LC2.1 | GSN | ATCTATNHSNHSNHSAATCGG | GAGGAGTTTGGGGGCTCTTC |
| | | (SEQ ID: | CCCTCAGGGGTC | CTG (SEQ ID NO. 33) |
| | | 94) | (SEQ ID NO. 32) | |
| LCDR3 | LC3.1 | QSYDS | TACTGCNHSNHSNHSNHSNHS | ATAATCAGCCTCATCCTCAGC |
| | | (SEQ ID: | AGCCTGAATGCTATATTCGGC | CTGG |
| | | 95) | GGAG | (SEQ ID NO. 35) |
| | | | (SEQ ID NO. 34) | |
| | LC3.2 | DSSLN | TCCTATNHSNHSNHSNHSNHS | CTGGCAGTAATAATCAGCCT |
| | | (SEQ ID: | GCTATATTCGGCGGAGGGAC | CATCCTC |
| | | 96) | C | (SEQ ID NO. 37) |
| | | | (SEQ ID NO. 36) | |

To identify specific CDR(s) that appear to have contributed more to GPC3 binding for further improved binding, soluble scFv ELISA was carried out with each library. Individual colonies were picked and seeded onto 96-well round-bottom plates containing 2x YT+100 μg/mL Carbenicillin+2% glucose for overnight amplification. After amplification, the overnight culture was seeded to fresh 2× YT+100 μg/mL Carbenicillin, induced with 1 mM IPTG, and treated with 5000 units/mL polymyxin B for scFv release. The clear supernatant containing soluble scFv was acquired by spinning down the cells.

The scFv ELISA was generally performed as follows. Immulon 4 HBX plates (Thermo Scientific, cat #3855) were coated with 1 μg/mL human GPC3 (Sino Biological, cat #10088-H08H) in PBS at 4° C. overnight. The plates were washed (3× with PBST (PBS+0.1% Tween-20), then the plates were blocked with 4% non-fat dry milk in PBST and incubated with a 1:1 dilution of supernatant containing scFv for 1 h (at RT). The bound scFvs were detected with 1:2000 diluted c-Myc antibody (Invitrogen, cat #MA1-980-1 MG) and 1:1000 diluted Mouse IgG HRP-conjugated secondary antibody (R&D Systems, cat #HAF007). Each incubation step was for 1 h at RT. Plates were washed three times with PBST between each incubation step. The color was developed by adding 1-Step™ TMB-ELISA substrate (Thermo Scientific, cat #34029), followed by the stop of reaction with 2 M sulfuric acid. Then OD₄₅₀ values were acquired by SpectraMax M3 (Molecular Devices, CA, USA).

Positive clones were defined as having OD₄₅₀ values 2-fold higher than that of the P17 WT, and were sequenced. The positive rate of each CDR is listed in Table 7. Heavy chain CDRs exhibited higher positive rates relative to light chain CDRs, indicating that mutations on heavy chain CDRs may benefit more than light chain CDRs from affinity maturation. From this point, the affinity maturation was principally focused on heavy chain CDRs.

TABLE 7
CDR and positive rates

| CDR | Colonies picked (cfu) | Positive rate (%) |
|---|---|---|
| HCDR1 | 94 | 14.9 |
| HCDR2 | 94 | 25.5 |
| HCDR3 | 94 | 12.8 |
| LCDR1 | 94 | 0.0 |
| LCDR2 | 94 | 3.2 |

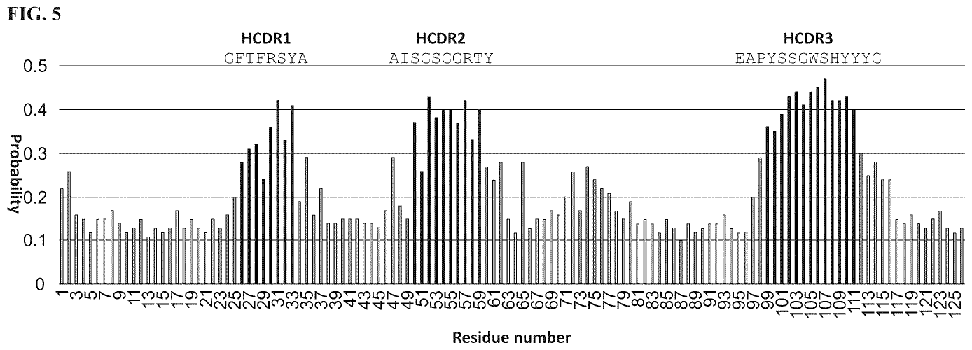

HCDR3 is generally known to play an important role in antigen recognition. The VH sequence of P17 was re-analyzed with proABC-2 online program available at the Utrecht Biomolecular Interaction Web Portal (available at the website: bianca.science.uu.nl/proabc2/). The webserver program estimates the probability that each residue interacts with the cognate antigen based on a deep learning framework. The prediction results are illustrated in FIG. 5. Two new HCDR3 libraries were constructed based on these predictive results and the sequencing results from the positive clones of the initial HCDR3 libraries. Two rounds of panning were performed, generally according to the procedure discussed above in this section. Clones identified by ELISA (high OD450 readings) were sequenced.

Another library was constructed focused on the beneficial mutations identified by the prior sequencing results, while diminishing the size of the library. Further, mutagenesis at HCDR1 and HCDR2 was performed in order to reduce potential immunogenicity of P17, with the overall aim of reversing the two CDRs back to germline sequences and improving antibody affinity. All degenerate oligos are listed in Table 8. To improve binding affinity, libraries of three heavy chain CDRs were combined in the following order: (1) HCDR1 and HCDR2 libraries were combined and panned against GPC3 for two rounds; (2) HCDR3 focused library after one round of panning was combined with the panned HCDR1-HCDR2 combined library; (3) the heavy chain shuffling library was panned against GPC3 for two more rounds. After that, over 600 individual colonies were picked from the HCDR3 focused library and the heavy chain shuffling library before and after the second round of panning. Soluble scFvs were produced and analyzed by ELISA using the same procedures described above. Of 56 sequenced clones, 9 clones (P17-8E12, P17-11F4, P17-13F3, P17-13G2, P17-15D5, P17-15F8, P17-16A1, P17-16A11, P17-16E6) with unique sequences and highest OD₄₅₀ readings in ELISA were selected as high affinity lead molecules.

TABLE 8
Oligonucleotides

| | | Target | | |
|---|---|---|---|---|
| CDR | Library | sequence* | Forward primer (5′→3′) | Reverse primer (5′→3′) |
| HCDR1 | HC1-3 | RSYA | GCAGCCTCTGGATTCACCTTT | TGAGACCCACTCCAGCCCCTT |
| | | (SEQ ID: 97) | RNSRVWTATGNCATGAGCTG | C |
| | | | GGTCCGCC | (SEQ ID NO. 39) |
| | | | (SEQ ID NO. 38) | |
| HCDR2 | HC2-3 | AISGSGGR | GGGCTGGAGTGGGTCTCARN | CCCATTCAGATCCTCTTCTGA |
| | | (SEQ ID: 98) | TNTSAGTRVTAGTGGTRNTRN | GATGAG |
| | | | SACATACTACGCAGACTCC | (SEQ ID NO. 41) |
| | | | (SEQ ID NO. 40) | |
| HCDR3 | HC3-4 | EAPYSS | AAAGAWNNSNNSNTSNNSNN | CGCACAGTAATATACGGCCG |
| | | (SEQ ID: 99) | SGGCTGGTCTCACTACTACTA | TG |
| | | | C | (SEQ ID NO. 43) |
| | | | (SEQ ID NO. 42) | |
| | HC3-5 | SGWSH | TATAGCNNSNGSNNSNNSNNS | CGGGGCTTCTTTCGCACAGTA |
| | | (SEQ ID: 100) | TACTACTACGGTATGGACGTCA | (SEQ ID NO. 45) |
| | | | TATAC (SEQ ID NO. 44) | |
| | focused | EAPYSSGWS | CCGTATATTACTGTGCGAAA | CCCATTCAGATCCTCTTCTGA |
| | | (SEQ ID: 101) | GAWNNSGACTWCNNSNNSGG | GATGAG |
| | | | CTGGWYGCACTACTAC | (SEQ ID NO. 47) |
| | | | (SEQ ID NO. 46) | |

### Example 10—GPC3 Binding of Affinity Matured Constructs

Antibody binding affinity of the affinity matured sequences was measured by the three-step protocol generally described above (i.e., with Octet QKe (ForteBio, Torrance, CA)). Step one prepares the anti-human Fc capture (AHC) biosensor probes by loading with testing antibody (at 2.5 ug/mL) and a reference biosensor probe with the same amount of a control antibody. Step two contacts the probes with serial dilutions of antigen (100 nM, 50 nM, 25 nM, 12.5 nM, 6.25 nM, 3.125 nM, and 0 nM GPC3, Sino Biological) for 600 seconds. An additional 100 nM antigen well was set up for the reference probe. The probes were then transferred into kinetics buffer solution (above) and allowed to dissociate for 300 seconds. The data were fitted to a 1:1 binding model using Octet data analysis software.

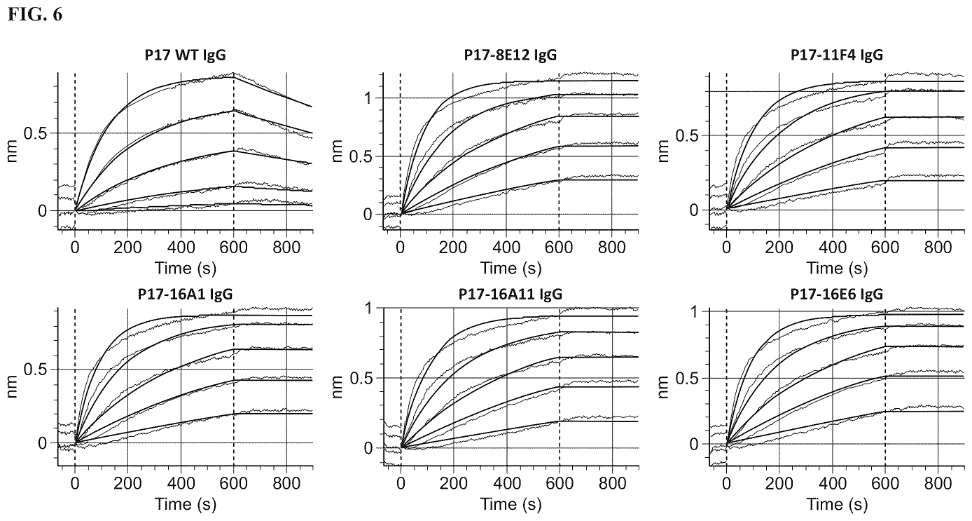

The binding kinetics of P17 WT and 5 affinity matured constructs are shown in FIG. 6 and the calculated K_{D}s are presented in Table 9. The affinity matured constructs exhibited substantially improved K_{D}s (i.e., orders of magnitude) which can correlate to significantly slower dissociation rates. For example, the K_{D}s of P17-8E12 and P17-11F4 reached the sub-nanomolar level, calculated as 6000 times lower than that of P17 WT.

TABLE 9
Binding of affinity matured constructs

| Loading sample | K_{D} (M) | Ka (1/Ms) | Kdis (1/s) |
|---|---|---|---|
| P17 WT IgG | 5.99E−09 | 1.39E+05 | 8.33E−04 |
| P17-8E12 IgG | <1.0E−12 | 2.23E+05 | 1.79E−07 |
| P17-11F4 IgG | <1.0E−12 | 1.88E+05 | 1.83E−07 |
| P17-16A1 IgG | 1.17E−12 | 2.15E+05 | 2.51E−07 |
| P17-16A11 IgG | 1.03E−12 | 1.82E+05 | 1.87E−07 |
| P17-16E6 IgG | 1.33E−12 | 2.08E+05 | 2.76E−07 |

ELISA was used to compare all the affinity matured constructs with P17 WT. Briefly, Immulon 4 HBX plates (Thermo Scientific, cat #3855) were coated with 1 μg/mL Fc-tagged human Glypican 3 protein (Acro Biosystems, cat #GP3-H5258-100 ug) in PBS at 4° C. overnight. After three washes with PBST (PBS+0.1% Tween-20), the plates were blocked with 4% non-fat dry milk in PBST and incubated with serial dilutions of the Fab constructs. The bound Fabs were detected with HRP-conjugated anti-Lambda light chain antibody (Abcam, cat #ab9007) at 1:1000 dilution. The incubation for each step was 1 h at RT. Plates were washed three times with PBST between each incubation step. The color was developed by adding 1-Step™ TMB-ELISA substrate (Thermo Scientific, cat #34029), followed by the stop reaction using 2 M sulfuric acid. OD₄₅₀ values were acquired by SpectraMax M3 (Molecular Devices, CA, USA).

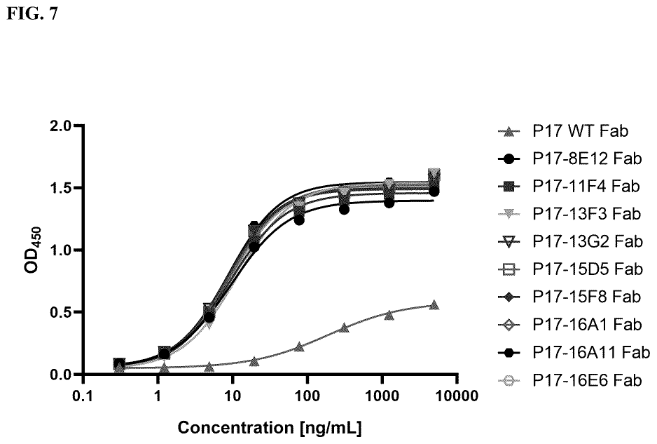

FIG. 7 shows the ELISA binding curves of titrated P17 WT and the 9 affinity matured constructs that were analyzed. All the constructs exhibited substantially lower EC50s (ranging between 8.4-11.7 ng/mL), and greater maximum responses when compared to P17 WT (Table 10). The results illustrate the improved binding capacity of the affinity matured constructs to recombinant human GPC3.

TABLE 10
EC50s of affinity matured constructs

| | | EC50 in cell surface |
|---|---|---|
| Sample | EC50 in ELISA(ng/mL) | binding(ng/mL) |
| P17 WT Fab | 191.3 | 21.3 |
| P17-8E12 Fab | 9.6 | 8.3 |
| P17-11F4 Fab | 9.5 | 9.9 |
| P17-13F3 Fab | 11.7 | 14.8 |
| P17-13G2 Fab | 8.4 | 13.0 |
| P17-15D5 Fab | 9.0 | 9.7 |
| P17-15F8 Fab | 8.9 | 10.4 |
| P17-16A1 Fab | 9.3 | 7.4 |
| P17-16A11 Fab | 8.7 | 8.3 |
| P17-16E6 Fab | 9.6 | 8.5 |

### Example 11—Cell Surface Binding of P17 Affinity Matured Constructs

The affinity matured P17 constructs were screened by flow cytometry for cell surface binding activity to endogenously expressed GPC3. Briefly, Hep3B cells (ATCC, cat #HB-8064) were dislodged from the culture flasks and blocked with PBS+1% FBS on ice for 1 hour. The cells were diluted (2×10⁵ cells/well) in a 96-well plate and stained with serial dilutions of P17 WT and affinity matured constructs, in the Fab format, on ice for 1 hour. After two washes with PBS+1% FBS, Fabs binding to the cell surface were detected with Alexa-647 labeled anti-human IgG (H+L) antibody (Invitrogen, cat #A21445, at 1:1000 dilution). The cells were washed twice and then stained with 1:1000 diluted SYTOX Green dead cell stain (Invitrogen, cat #S34860) on ice to differentiate the dead cells from the live ones. After removing the free dye by two rounds of wash, the cells were analyzed on flow cytometer.

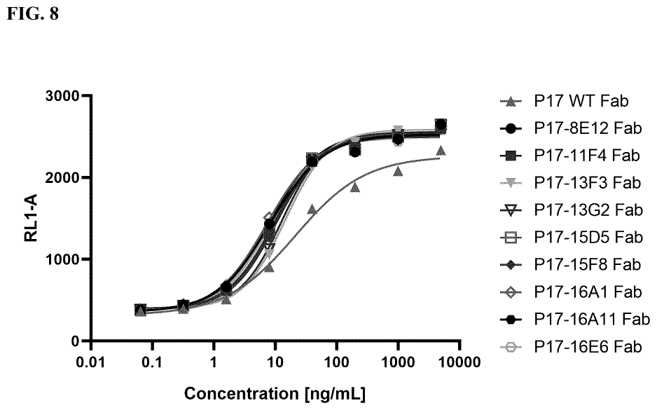

The cell surface binding profiles shown in FIG. 8 demonstrate that the affinity matured P17 constructs bind to GPC3 expressed on the cell surface is improved relative to P17 WT, with the majority of constructs exhibiting EC50 values below 10 ng/ml (Table 10, above).

A breast cancer cell line (T-47D, ATCC, cat #HTB-133) which is not known to express GPC3, was used to assess whether the affinity matured constructs developed any non-specific binding capacity during the maturation process. T-47D cells were dislodged from the culture flask and blocked with PBS+1% FBS on ice for 1 hour. The cells were seeded at 2×10⁵ cells/well into a 96-well plate and stained with P17 WT and affinity matured constructs (as IgGs) at a concentration of 9 μg/mL. Rituximab, an FDA-approved drug known to specifically bind to CD22, served as negative control in the experiment. The bound IgGs were detected with a PE-conjugated Goat anti-Human IgG Fc secondary antibody (Invitrogen, cat #12-4998-82) at 1:1000 dilution. Dead cells were stained with LIVE/DEAD™ Fixable Far Red (Invitrogen, cat #L34973) at 1:1000 dilution. All incubation steps were performed on ice for one hour. Between each incubation step, cells were washed twice with PBS+1% FBS to remove any free antibody or dye. The fluorescence signal was detected on a flow cytometer.

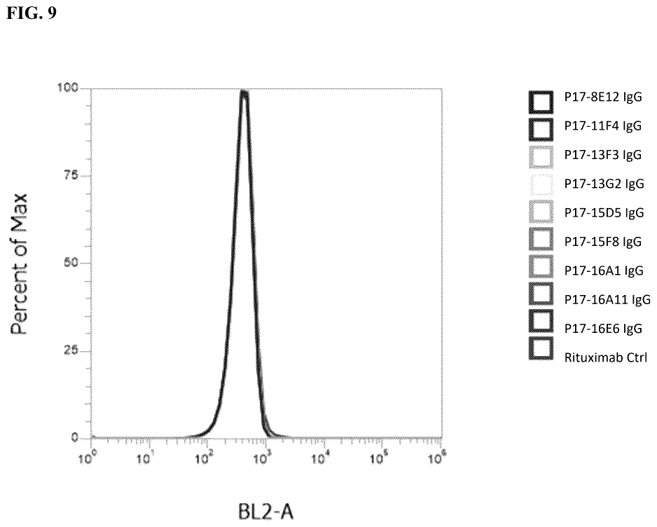

The histograms of all nine candidates overlapped with that of the negative control, indicating that none of the candidates possessed any non-specific binding activity to GPC3-negative T-47D cells. (FIG. 9)

TABLE 11
Heavy Chain Variable Region Amino Acid Sequences & CDRs for Representative
Affinity Matured GPC3 Constructs.

| Internal | | | | |
|---|---|---|---|---|
| Ref. | VH sequence | CDR1 | CDR2 | CDR3 |
| P17_8E1 | EVQLVESGGGLVQPGGSLRL | GFTFRSYA | ISGSGGRT | AKEYDFKTGWM |
| 2_VH | SCAASGFTFRSYAMSWVRQ | (SEQ ID: 49) | (SEQ ID: 50) | HYYYGMDV |
| | APGKGLEWVSAISGSGGRTY | | | (SEQ ID: 51) |
| | YADSVKGRFTISRDNSKNTL | | | |
| | YLQMNSLRAEDTAVYYCAK | | | |
| | EYDFKTGWMHYYYGMDVW | | | |
| | GQGTTVTVSS | | | |
| | (SEQ ID: 48) | | | |
| P17_11F | EVQLVESGGGLVQPGGSLRL | GFTFKDYA | LSGSGGAT | AKEMDFRYGW |
| 4_VH | SCAASGFTFKDYAMSWVRQ | (SEQ ID: 53) | (SEQ ID: 54) | MHYYYGMDV |
| | APGKGLEWVSALSGSGGAT | | | (SEQ ID: 55) |
| | YYADSVKGRFTISRDNSKNT | | | |
| | LYLQMNSLRAEDTAVYYCA | | | |
| | KEMDFRYGWMHYYYGMDV | | | |
| | WGQGTTVTVSS | | | |
| | (SEQ ID: 52) | | | |
| P17_13F | EVQLVESGGGLVQPGGSLRL | GFTFEGYA | VSGSGDGT | AKEYDYKTGW |
| 3_VH | SCAASGFTFEGYAMSWVRQ | (SEQ ID: 57) | (SEQ ID: 58) | MHYYYGMDV |
| | APGKGLEWVSNVSGSGDGT | | | (SEQ ID: 59) |
| | YYADSVKGRFTISRDNSKNT | | | |
| | LYLQMNSLRAEDTAVYYCA | | | |
| | KEYDYKTGWMHYYYGMDV | | | |
| | WGQGTTVTVSS | | | |
| | (SEQ ID: 56) | | | |
| P17_13G | EVQLVESGGGLVQPGGSLRL | GFTFGEYA | MSGSGDGT | AKEMDYQLGW |
| 2_VH | SCAASGFTFGEYAMSWVRQ | (SEQ ID: 61) | (SEQ ID: 62) | MHYYYGMDV |
| | APGKGLEWVSGMSGSGDGT | | | (SEQ ID: 63) |
| | YYADSVKGRFTISRDNSKNT | | | |
| | LYLQMNSLRAEDTAVYYCA | | | |
| | KEMDYQLGWMHYYYGMDV | | | |
| | WGQGTTVTVSS | | | |
| | (SEQ ID: 60) | | | |
| P17_15D | EVQLVESGGGLVQPGGSLRL | GFTFANYA | VSASGVGT | AKEVDFKLGWM |
| 5_VH | SCAASGFTFANYAMSWVRQ | (SEQ ID: 65) | (SEQ ID: 66) | HYYYGMDV |
| | APGKGLEWVSDVSASGVGT | | | (SEQ ID: 67) |
| | YYADSVKGRFTISRDNSKNT | | | |
| | LYLQMNSLRAEDTAVYYCA | | | |
| | KEVDFKLGWMHYYYGMDV | | | |
| | WGQGTTVTVSS | | | |
| | (SEQ ID: 64) | | | |
| P17_15F | EVQLVESGGGLVQPGGSLRL | GFTFGNYA | VSGSGDGT | AKEYDFGLGWM |
| 8_VH | SCAASGFTFGNYAMSWVRQ | (SEQ ID: 69) | (SEQ ID: 70) | HYYYGMDV |
| | APGKGLEWVSGVSGSGDGT | | | (SEQ ID: 71) |
| | YYADSVKGRFTISRDNSKNT | | | |
| | LYLQMNSLRAEDTAVYYCA | | | |
| | KEYDFGLGWMHYYYGMDV | | | |
| | WGQGTTVTVSS | | | |
| | (SEQ ID: 68) | | | |
| P17_16A | EVQLVESGGGLVQPGGSLRL | GFTFKRYA | VSSSGTGT | AKEYDYGLGW |
| 1_VH | SCAASGFTFKRYAMSWVRQ | (SEQ ID: 73) | (SEQ ID: 74) | MHYYYGMDV |
| | APGKGLEWVSSVSSSGTGTY | | | (SEQ ID: 75) |
| | YADSVKGRFTISRDNSKNTL | | | |
| | YLQMNSLRAEDTAVYYCAK | | | |
| | EYDYGLGWMHYYYGMDV | | | |
| | WGQGTTVTVSS | | | |
| | (SEQ ID: 72) | | | |
| P17_16A | EVQLVESGGGLVQPGGSLRL | GFTFDRYA | LSASGSGT | AKEYDFSYGWM |
| 11_VH | SCAASGFTFDRYAMSWVRQ | (SEQ ID: 77) | (SEQ ID: 78) | HYYYGMDV |
| | APGKGLEWVSSLSASGSGTY | | | (SEQ ID: 79) |
| | YADSVKGRFTISRDNSKNTL | | | |
| | YLQMNSLRAEDTAVYYCAK | | | |
| | EYDFSYGWMHYYYGMDVW | | | |
| | GQGTTVTVSS | | | |
| | (SEQ ID: 76) | | | |
| P17_16E | EVQLVESGGGLVQPGGSLRL | GFTFRKYA | LSASGSAT | AKEADYSMGW |
| 6_VH | SCAASGFTFRKYAMSWVRQ | (SEQ ID: 81) | (SEQ ID: 82) | MHYYYGMDV |
| | APGKGLEWVSSLSASGSATY | | | (SEQ ID: 83) |
| | YADSVKGRFTISRDNSKNTL | | | |
| | YLQMNSLRAEDTAVYYCAK | | | |
| | EADYSMGWMHYYYGMDV | | | |
| | WGQGTTVTVSS | | | |
| | (SEQ ID: 80) | | | |

We claim:

1. An isolated antibody or antigen-binding fragment thereof that binds to glypican 3 (GPC3) protein, the antibody or antigen-binding fragment thereof comprising:
i. a heavy chain variable region comprising: a CDR1, a CDR2, and a CDR3 of the amino acid sequence of SEQ ID NO: 1; and
a light chain variable region comprising: a CDR1, a CDR2, and a CDR3 amino acid sequences of SEQ ID NO: 2;
ii. a heavy chain variable region comprising: a CDR1, a CDR2, and a CDR3 of the amino acid sequence of SEQ ID NO: 3; and
a light chain variable region comprising: a CDR1, a CDR2, and a CDR3 amino acid sequences of SEQ ID NO: 4;
iii. a heavy chain variable region comprising: a CDR1, a CDR2, and a CDR3 of the amino acid sequence of SEQ ID NO: 5; and
a light chain variable region comprising: a CDR1, a CDR2, and a CDR3 amino acid sequences of SEQ ID NO: 6;
iv. a heavy chain variable region comprising: a CDR1, a CDR2, and a CDR3 of the amino acid sequence of SEQ ID NO: 7; and
a light chain variable region comprising: a CDR1, a CDR2, and a CDR3 amino acid sequences of SEQ ID NO: 8;
v. a heavy chain variable region comprising: a CDR1, a CDR2, and a CDR3 of the amino acid sequence of SEQ ID NO: 9; and
a light chain variable region comprising: a CDR1, a CDR2, and a CDR3 amino acid sequences of SEQ ID NO: 10;
vi. a heavy chain variable region comprising: a CDR1, a CDR2, and a CDR3 of the amino acid sequence of SEQ ID NO: 11; and
a light chain variable region comprising: a CDR1, a CDR2, and a CDR3 amino acid sequences of SEQ ID NO: 12;
vii. a heavy chain variable region comprising: a CDR1, a CDR2, and a CDR3 of the amino acid sequence of SEQ ID NO: 13; and
a light chain variable region comprising: a CDR1, a CDR2, and a CDR3 amino acid sequences of SEQ ID NO: 14;
viii. a heavy chain variable region comprising: a CDR1, a CDR2, and a CDR3 of the amino acid sequence of SEQ ID NO: 15; and
a light chain variable region comprising: a CDR1, a CDR2, and a CDR3 amino acid sequences of SEQ ID NO: 16;
ix. a heavy chain variable region comprising: a CDR1, a CDR2, and a CDR3 of the amino acid sequence of SEQ ID NO: 48; and
a light chain variable region comprising: a CDR1, a CDR2, and a CDR3 amino acid sequences of SEQ ID NO: 8;
x. a heavy chain variable region comprising: a CDR1, a CDR2, and a CDR3 of the amino acid sequence of SEQ ID NO: 52; and
a light chain variable region comprising: a CDR1, a CDR2, and a CDR3 amino acid sequences of SEQ ID NO: 8;
xi. a heavy chain variable region comprising: a CDR1, a CDR2, and a CDR3 of the amino acid sequence of SEQ ID NO: 56; and
a light chain variable region comprising: a CDR1, a CDR2, and a CDR3 amino acid sequences of SEQ ID NO: 8;
xii. a heavy chain variable region comprising: a CDR1, a CDR2, and a CDR3 of the amino acid sequence of SEQ ID NO: 60; and
a light chain variable region comprising: a CDR1, a CDR2, and a CDR3 amino acid sequences of SEQ ID NO: 8;
xiii. a heavy chain variable region comprising: a CDR1, a CDR2, and a CDR3 of the amino acid sequence of SEQ ID NO: 64; and
a light chain variable region comprising: a CDR1, a CDR2, and a CDR3 amino acid sequences of SEQ ID NO: 8;
xiv. a heavy chain variable region comprising: a CDR1, a CDR2, and a CDR3 of the amino acid sequence of SEQ ID NO: 68; and
a light chain variable region comprising: a CDR1, a CDR2, and a CDR3 amino acid sequences of SEQ ID NO: 8;
xv. a heavy chain variable region comprising: a CDR1, a CDR2, and a CDR3 of the amino acid sequence of SEQ ID NO: 72; and
a light chain variable region comprising: a CDR1, a CDR2, and a CDR3 amino acid sequences of SEQ ID NO: 8;
xvi. a heavy chain variable region comprising: a CDR1, a CDR2, and a CDR3 of the amino acid sequence of SEQ ID NO: 76; and
a light chain variable region comprising: a CDR1, a CDR2, and a CDR3 amino acid sequences of SEQ ID NO: 8; or
xvii. a heavy chain variable region comprising: a CDR1, a CDR2, and a CDR3 of the amino acid sequence of SEQ ID NO: 80; and
a light chain variable region comprising: a CDR1, a CDR2, and a CDR3 amino acid sequences of SEQ ID NO: 8.

2. The isolated antibody or antigen-binding fragment thereof of claim 1, comprising:
i. a heavy chain variable region comprising SEQ ID NO: 1; and
a light chain variable region comprising SEQ ID NO: 2;
ii. a heavy chain variable region comprising SEQ ID NO: 3; and
a light chain variable region comprising SEQ ID NO: 4;
iii. a heavy chain variable region comprising SEQ ID NO: 5; and
a light chain variable region comprising SEQ ID NO: 6;
iv. a heavy chain variable region comprising SEQ ID NO: 7; and
a light chain variable region comprising SEQ ID NO: 8;
v. a heavy chain variable region comprising SEQ ID NO: 9; and
a light chain variable region comprising SEQ ID NO: 10;
vi. a heavy chain variable region comprising SEQ ID NO: 11; and
a light chain variable region comprising SEQ ID NO: 12;
vii. a heavy chain variable region comprising SEQ ID NO: 13; and
a light chain variable region comprising SEQ ID NO: 14;
viii. a heavy chain variable region comprising SEQ ID NO: 15; and
a light chain variable region comprising SEQ ID NO: 16;
ix. a heavy chain variable region comprising SEQ ID NO: 48; and
a light chain variable region comprising SEQ ID NO: 8;
x. a heavy chain variable region comprising SEQ ID NO: 52; and
a light chain variable region comprising SEQ ID NO: 8;
xi. a heavy chain variable region comprising SEQ ID NO: 56; and
a light chain variable region comprising SEQ ID NO: 8;
xii. a heavy chain variable region comprising SEQ ID NO: 60; and
a light chain variable region comprising SEQ ID NO: 8;
xiii. a heavy chain variable region comprising SEQ ID NO: 64; and
a light chain variable region comprising SEQ ID NO: 8;
xiv. a heavy chain variable region comprising SEQ ID NO: 68; and
a light chain variable region comprising SEQ ID NO: 8;
xv. a heavy chain variable region comprising SEQ ID NO: 72; and
a light chain variable region comprising SEQ ID NO: 8;
xvi. a heavy chain variable region comprising SEQ ID NO: 76; and
a light chain variable region comprising SEQ ID NO: 8; or
xvii. a heavy chain variable region comprising SEQ ID NO: 80; and
a light chain variable region comprising SEQ ID NO: 8.

3. The isolated antibody or antigen-binding fragment thereof of any one of claims 1-2, wherein the antigen-binding fragment comprises a single-chain Fvs (scFv), a single chain Fv-Fc (scFv-Fc), a single-chain antibody, a single domain antibody, a Fab fragment, or a F(ab′)2 fragment.

4. The isolated antibody or antigen-binding fragment thereof of any one of claims 1-2, wherein the antibody comprises an IgG, an IgM, an IgA, an IgE, an IgD, or an IgY isotype.

5. The isolated antibody or antigen-binding fragment thereof of any one of claim 1, 2, or 4, wherein the antibody is a monoclonal antibody (mAb).

6. The isolated antibody or antigen-binding fragment thereof of any one of claims 1-5 that further comprises a conjugated moiety.

7. The isolated antibody or antigen-binding fragment thereof of claim 6, wherein the conjugated moiety comprises a therapeutic agent, a solid support, an affinity agent, or a detectable label.

8. The isolated antibody or antigen-binding fragment thereof of any one of claims 6-7 wherein the conjugated moiety comprises an anti-cancer agent or a cytotoxin.

9. The isolated antibody or antigen-binding fragment thereof of any one of claims 1-8 that binds to the glypican 3 protein with a K_{D} of from about 1.0 pM to 200 nM.

10. The isolated antibody or antigen-binding fragment thereof of any one of claims 1-8 that inhibits cell proliferation with an IC50 of from about 0.01-250 nM.

11. A bispecific antibody comprising the antibody or antigen-binding fragment thereof of any one of claims 1-5, and an antibody or antigen-binding fragment thereof that specifically binds to an antigen that does not comprise a GPC3 epitope.

12. The bispecific antibody of claim 11, wherein the antibody or antigen-binding fragment thereof that specifically binds to an antigen that does not comprise a GPC3 epitope comprises a therapeutic antibody.

13. The bispecific antibody of claim 11, wherein the antibody or antigen-binding fragment thereof that specifically binds to an antigen that does not comprise a GPC3 epitope induces an immune response.

14. The bispecific antibody of claim 11, wherein the antibody or antigen-binding fragment thereof that specifically binds to an antigen that does not comprise a GPC3 epitope binds to an antigen comprising CD2, CD3, CD11a CD20, CD25 (IL2R), CD28, CD33, CD47, CD52, EGFR, VEGF, Integrin-alpha 3, GPIIb/IIIar, Protein F, TNF-alpha, TNF-beta, HER2/Neu, C5, or IgE.

15. The isolated antibody or antigen-binding fragment thereof of any one of claims 1-5 that is conjugated to a solid support, an affinity agent, or a detectable agent.

16. A pharmaceutical composition comprising the isolated antibody or antigen-binding fragment thereof of any one of claims 1-15, and a pharmaceutically acceptable carrier.

17. A kit comprising the pharmaceutical composition of claim 16, or the antibody or antigen-binding fragment thereof of any one of claims 1-15, optional reagents, and instructions for use.

18. A method for treating cancer in a subject in need thereof, the method comprising administering to the subject an effective amount of an antibody or antigen-binding fragment thereof that binds to glypican 3 (GPC3) protein, the antibody or antigen-binding fragment thereof comprising:
i. a heavy chain variable region comprising: a CDR1, a CDR2, and a CDR3 of the amino acid sequence of SEQ ID NO: 1; and
a light chain variable region comprising: a CDR1, a CDR2, and a CDR3 amino acid sequences of SEQ ID NO: 2;
ii. a heavy chain variable region comprising: a CDR1, a CDR2, and a CDR3 of the amino acid sequence of SEQ ID NO: 3; and
a light chain variable region comprising: a CDR1, a CDR2, and a CDR3 amino acid sequences of SEQ ID NO: 4;
iii. a heavy chain variable region comprising: a CDR1, a CDR2, and a CDR3 of the amino acid sequence of SEQ ID NO: 5; and
a light chain variable region comprising: a CDR1, a CDR2, and a CDR3 amino acid sequences of SEQ ID NO: 6;
iv. a heavy chain variable region comprising: a CDR1, a CDR2, and a CDR3 of the amino acid sequence of SEQ ID NO: 7; and
a light chain variable region comprising: a CDR1, a CDR2, and a CDR3 amino acid sequences of SEQ ID NO: 8;
v. a heavy chain variable region comprising: a CDR1, a CDR2, and a CDR3 of the amino acid sequence of SEQ ID NO: 9; and
a light chain variable region comprising: a CDR1, a CDR2, and a CDR3 amino acid sequences of SEQ ID NO: 10;
vi. a heavy chain variable region comprising: a CDR1, a CDR2, and a CDR3 of the amino acid sequence of SEQ ID NO: 11; and
a light chain variable region comprising: a CDR1, a CDR2, and a CDR3 amino acid sequences of SEQ ID NO: 12;
vii. a heavy chain variable region comprising: a CDR1, a CDR2, and a CDR3 of the amino acid sequence of SEQ ID NO: 13; and
a light chain variable region comprising: a CDR1, a CDR2, and a CDR3 amino acid sequences of SEQ ID NO: 14;
viii. a heavy chain variable region comprising: a CDR1, a CDR2, and a CDR3 of the amino acid sequence of SEQ ID NO: 15; and
a light chain variable region comprising: a CDR1, a CDR2, and a CDR3 amino acid sequences of SEQ ID NO: 16;
ix. a heavy chain variable region comprising: a CDR1, a CDR2, and a CDR3 of the amino acid sequence of SEQ ID NO: 48; and
a light chain variable region comprising: a CDR1, a CDR2, and a CDR3 amino acid sequences of SEQ ID NO: 8;
x. a heavy chain variable region comprising: a CDR1, a CDR2, and a CDR3 of the amino acid sequence of SEQ ID NO: 52; and
a light chain variable region comprising: a CDR1, a CDR2, and a CDR3 amino acid sequences of SEQ ID NO: 8;
xi. a heavy chain variable region comprising: a CDR1, a CDR2, and a CDR3 of the amino acid sequence of SEQ ID NO: 56; and
a light chain variable region comprising: a CDR1, a CDR2, and a CDR3 amino acid sequences of SEQ ID NO: 8;
xii. a heavy chain variable region comprising: a CDR1, a CDR2, and a CDR3 of the amino acid sequence of SEQ ID NO: 60; and
a light chain variable region comprising: a CDR1, a CDR2, and a CDR3 amino acid sequences of SEQ ID NO: 8;
xiii. a heavy chain variable region comprising: a CDR1, a CDR2, and a CDR3 of the amino acid sequence of SEQ ID NO: 64; and
a light chain variable region comprising: a CDR1, a CDR2, and a CDR3 amino acid sequences of SEQ ID NO: 8;
xiv. a heavy chain variable region comprising: a CDR1, a CDR2, and a CDR3 of the amino acid sequence of SEQ ID NO: 68; and
a light chain variable region comprising: a CDR1, a CDR2, and a CDR3 amino acid sequences of SEQ ID NO: 8;
xv. a heavy chain variable region comprising: a CDR1, a CDR2, and a CDR3 of the amino acid sequence of SEQ ID NO: 72; and
a light chain variable region comprising: a CDR1, a CDR2, and a CDR3 amino acid sequences of SEQ ID NO: 8;
xvi. a heavy chain variable region comprising: a CDR1, a CDR2, and a CDR3 of the amino acid sequence of SEQ ID NO: 76; and
a light chain variable region comprising: a CDR1, a CDR2, and a CDR3 amino acid sequences of SEQ ID NO: 8; or
xvii. a heavy chain variable region comprising: a CDR1, a CDR2, and a CDR3 of the amino acid sequence of SEQ ID NO: 80; and
a light chain variable region comprising: a CDR1, a CDR2, and a CDR3 amino acid sequences of SEQ ID NO: 8.

19. The method according to claim 18, wherein the antibody or antigen-binding fragment thereof of claim 1, comprises:
i. a heavy chain variable region comprising SEQ ID NO: 1; and
a light chain variable region comprising SEQ ID NO: 2;
ii. a heavy chain variable region comprising SEQ ID NO: 3; and
a light chain variable region comprising SEQ ID NO: 4;
iii. a heavy chain variable region comprising SEQ ID NO: 5; and
a light chain variable region comprising SEQ ID NO: 6;
iv. a heavy chain variable region comprising SEQ ID NO: 7; and
a light chain variable region comprising SEQ ID NO: 8;
v. a heavy chain variable region comprising SEQ ID NO: 9; and
a light chain variable region comprising SEQ ID NO: 10;
vi. a heavy chain variable region comprising SEQ ID NO: 11; and
a light chain variable region comprising SEQ ID NO: 12;
vii. a heavy chain variable region comprising SEQ ID NO: 13; and
a light chain variable region comprising SEQ ID NO: 14;
viii. a heavy chain variable region comprising SEQ ID NO: 15; and
a light chain variable region comprising SEQ ID NO: 16;
ix. a heavy chain variable region comprising SEQ ID NO: 48; and
a light chain variable region comprising SEQ ID NO: 8;
x. a heavy chain variable region comprising SEQ ID NO: 52; and
a light chain variable region comprising SEQ ID NO: 8;
xi. a heavy chain variable region comprising SEQ ID NO: 56; and
a light chain variable region comprising SEQ ID NO: 8;
xii. a heavy chain variable region comprising SEQ ID NO: 60; and
a light chain variable region comprising SEQ ID NO: 8;
xiii. a heavy chain variable region comprising SEQ ID NO: 64; and
a light chain variable region comprising SEQ ID NO: 8;
xiv. a heavy chain variable region comprising SEQ ID NO: 68; and
a light chain variable region comprising SEQ ID NO: 8;
xv. a heavy chain variable region comprising SEQ ID NO: 72; and
a light chain variable region comprising SEQ ID NO: 8;
xvi. a heavy chain variable region comprising SEQ ID NO: 76; and
a light chain variable region comprising SEQ ID NO: 8; or
xvii. a heavy chain variable region comprising SEQ ID NO: 80; and
a light chain variable region comprising SEQ ID NO: 8.

20. The method according to any of claims 18-19, wherein the cancer comprises a solid tumor cell cancer.

21. The method according to any of claims 18-20, wherein the cancer comprises a liver cancer.

22. A method inhibiting proliferation of a cell that expresses glypican 3 protein, the method comprising contacting the cell with an effective amount of an antibody or antigen-binding fragment thereof that binds to glypican 3 (GPC3) protein, the antibody or antigen-binding fragment thereof comprising:
i. a heavy chain variable region comprising: a CDR1, a CDR2, and a CDR3 of the amino acid sequence of SEQ ID NO: 1; and
a light chain variable region comprising: a CDR1, a CDR2, and a CDR3 amino acid sequences of SEQ ID NO: 2;
ii. a heavy chain variable region comprising: a CDR1, a CDR2, and a CDR3 of the amino acid sequence of SEQ ID NO: 3; and
a light chain variable region comprising: a CDR1, a CDR2, and a CDR3 amino acid sequences of SEQ ID NO: 4;
iii. a heavy chain variable region comprising: a CDR1, a CDR2, and a CDR3 of the amino acid sequence of SEQ ID NO: 5; and
a light chain variable region comprising: a CDR1, a CDR2, and a CDR3 amino acid sequences of SEQ ID NO: 6;
iv. a heavy chain variable region comprising: a CDR1, a CDR2, and a CDR3 of the amino acid sequence of SEQ ID NO: 7; and
a light chain variable region comprising: a CDR1, a CDR2, and a CDR3 amino acid sequences of SEQ ID NO: 8;
v. a heavy chain variable region comprising: a CDR1, a CDR2, and a CDR3 of the amino acid sequence of SEQ ID NO: 9; and
a light chain variable region comprising: a CDR1, a CDR2, and a CDR3 amino acid sequences of SEQ ID NO: 10;
vi. a heavy chain variable region comprising: a CDR1, a CDR2, and a CDR3 of the amino acid sequence of SEQ ID NO: 11; and
a light chain variable region comprising: a CDR1, a CDR2, and a CDR3 amino acid sequences of SEQ ID NO: 12;
vii. a heavy chain variable region comprising: a CDR1, a CDR2, and a CDR3 of the amino acid sequence of SEQ ID NO: 13; and
a light chain variable region comprising: a CDR1, a CDR2, and a CDR3 amino acid sequences of SEQ ID NO: 14;
viii. a heavy chain variable region comprising: a CDR1, a CDR2, and a CDR3 of the amino acid sequence of SEQ ID NO: 15; and
a light chain variable region comprising: a CDR1, a CDR2, and a CDR3 amino acid sequences of SEQ ID NO: 16;
ix. a heavy chain variable region comprising: a CDR1, a CDR2, and a CDR3 of the amino acid sequence of SEQ ID NO: 48; and
a light chain variable region comprising: a CDR1, a CDR2, and a CDR3 amino acid sequences of SEQ ID NO: 8;
x. a heavy chain variable region comprising: a CDR1, a CDR2, and a CDR3 of the amino acid sequence of SEQ ID NO: 52; and
a light chain variable region comprising: a CDR1, a CDR2, and a CDR3 amino acid sequences of SEQ ID NO: 8;
xi. a heavy chain variable region comprising: a CDR1, a CDR2, and a CDR3 of the amino acid sequence of SEQ ID NO: 56; and
a light chain variable region comprising: a CDR1, a CDR2, and a CDR3 amino acid sequences of SEQ ID NO: 8;
xii. a heavy chain variable region comprising: a CDR1, a CDR2, and a CDR3 of the amino acid sequence of SEQ ID NO: 60; and
a light chain variable region comprising: a CDR1, a CDR2, and a CDR3 amino acid sequences of SEQ ID NO: 8;
xiii. a heavy chain variable region comprising: a CDR1, a CDR2, and a CDR3 of the amino acid sequence of SEQ ID NO: 64; and
a light chain variable region comprising: a CDR1, a CDR2, and a CDR3 amino acid sequences of SEQ ID NO: 8;
xiv. a heavy chain variable region comprising: a CDR1, a CDR2, and a CDR3 of the amino acid sequence of SEQ ID NO: 68; and
a light chain variable region comprising: a CDR1, a CDR2, and a CDR3 amino acid sequences of SEQ ID NO: 8;
xv. a heavy chain variable region comprising: a CDR1, a CDR2, and a CDR3 of the amino acid sequence of SEQ ID NO: 72; and
a light chain variable region comprising: a CDR1, a CDR2, and a CDR3 amino acid sequences of SEQ ID NO: 8;
xvi. a heavy chain variable region comprising: a CDR1, a CDR2, and a CDR3 of the amino acid sequence of SEQ ID NO: 76; and
a light chain variable region comprising: a CDR1, a CDR2, and a CDR3 amino acid sequences of SEQ ID NO: 8; or
xvii. a heavy chain variable region comprising: a CDR1, a CDR2, and a CDR3 of the amino acid sequence of SEQ ID NO: 80; and
a light chain variable region comprising: a CDR1, a CDR2, and a CDR3 amino acid sequences of SEQ ID NO: 8.

23. The method according to claim 22, wherein the antibody or antigen-binding fragment thereof of claim 1, comprises:
i. a heavy chain variable region comprising SEQ ID NO: 1; and
a light chain variable region comprising SEQ ID NO: 2;
ii. a heavy chain variable region comprising SEQ ID NO: 3; and
a light chain variable region comprising SEQ ID NO: 4;
iii. a heavy chain variable region comprising SEQ ID NO: 5; and
a light chain variable region comprising SEQ ID NO: 6;
iv. a heavy chain variable region comprising SEQ ID NO: 7; and
a light chain variable region comprising SEQ ID NO: 8;
v. a heavy chain variable region comprising SEQ ID NO: 9; and
a light chain variable region comprising SEQ ID NO: 10;
vi. a heavy chain variable region comprising SEQ ID NO: 11; and
a light chain variable region comprising SEQ ID NO: 12;
vii. a heavy chain variable region comprising SEQ ID NO: 13; and
a light chain variable region comprising SEQ ID NO: 14;
viii. a heavy chain variable region comprising SEQ ID NO: 15; and
a light chain variable region comprising SEQ ID NO: 16;
ix. a heavy chain variable region comprising SEQ ID NO: 48; and
a light chain variable region comprising SEQ ID NO: 8;
x. a heavy chain variable region comprising SEQ ID NO: 52; and
a light chain variable region comprising SEQ ID NO: 8;
xi. a heavy chain variable region comprising SEQ ID NO: 56; and
a light chain variable region comprising SEQ ID NO: 8;
xii. a heavy chain variable region comprising SEQ ID NO: 60; and
a light chain variable region comprising SEQ ID NO: 8;
xiii. a heavy chain variable region comprising SEQ ID NO: 64; and
a light chain variable region comprising SEQ ID NO: 8;
xiv. a heavy chain variable region comprising SEQ ID NO: 68; and
a light chain variable region comprising SEQ ID NO: 8;
xv. a heavy chain variable region comprising SEQ ID NO: 72; and
a light chain variable region comprising SEQ ID NO: 8;
xvi. a heavy chain variable region comprising SEQ ID NO: 76; and
a light chain variable region comprising SEQ ID NO: 8; or
xvii. a heavy chain variable region comprising SEQ ID NO: 80; and
a light chain variable region comprising SEQ ID NO: 8.

24. The method according to any of claims 22-23, wherein the cell comprises a cancer cell.

25. The method according to any of claim 24, wherein the cell comprises a liver cancer cell.

26. A method of detecting the presence of glypican 3 (GPC3) protein in a biological sample, comprising contacting the biological sample with an effective amount of an antibody or antigen-binding fragment thereof that binds to glypican 3 (GPC3) protein, the antibody or antigen-binding fragment thereof comprising:
i. a heavy chain variable region comprising: a CDR1, a CDR2, and a CDR3 of the amino acid sequence of SEQ ID NO: 1; and
a light chain variable region comprising: a CDR1, a CDR2, and a CDR3 amino acid sequences of SEQ ID NO: 2;
ii. a heavy chain variable region comprising: a CDR1, a CDR2, and a CDR3 of the amino acid sequence of SEQ ID NO: 3; and
a light chain variable region comprising: a CDR1, a CDR2, and a CDR3 amino acid sequences of SEQ ID NO: 4;
iii. a heavy chain variable region comprising: a CDR1, a CDR2, and a CDR3 of the amino acid sequence of SEQ ID NO: 5; and
a light chain variable region comprising: a CDR1, a CDR2, and a CDR3 amino acid sequences of SEQ ID NO: 6;
iv. a heavy chain variable region comprising: a CDR1, a CDR2, and a CDR3 of the amino acid sequence of SEQ ID NO: 7; and
a light chain variable region comprising: a CDR1, a CDR2, and a CDR3 amino acid sequences of SEQ ID NO: 8;
v. a heavy chain variable region comprising: a CDR1, a CDR2, and a CDR3 of the amino acid sequence of SEQ ID NO: 9; and
a light chain variable region comprising: a CDR1, a CDR2, and a CDR3 amino acid sequences of SEQ ID NO: 10;
vi. a heavy chain variable region comprising: a CDR1, a CDR2, and a CDR3 of the amino acid sequence of SEQ ID NO: 11; and
a light chain variable region comprising: a CDR1, a CDR2, and a CDR3 amino acid sequences of SEQ ID NO: 12;
vii. a heavy chain variable region comprising: a CDR1, a CDR2, and a CDR3 of the amino acid sequence of SEQ ID NO: 13; and
a light chain variable region comprising: a CDR1, a CDR2, and a CDR3 amino acid sequences of SEQ ID NO: 14;
viii. a heavy chain variable region comprising: a CDR1, a CDR2, and a CDR3 of the amino acid sequence of SEQ ID NO: 15; and
a light chain variable region comprising: a CDR1, a CDR2, and a CDR3 amino acid sequences of SEQ ID NO: 16;
ix. a heavy chain variable region comprising: a CDR1, a CDR2, and a CDR3 of the amino acid sequence of SEQ ID NO: 48; and
a light chain variable region comprising: a CDR1, a CDR2, and a CDR3 amino acid sequences of SEQ ID NO: 8;
x. a heavy chain variable region comprising: a CDR1, a CDR2, and a CDR3 of the amino acid sequence of SEQ ID NO: 52; and
a light chain variable region comprising: a CDR1, a CDR2, and a CDR3 amino acid sequences of SEQ ID NO: 8;
xi. a heavy chain variable region comprising: a CDR1, a CDR2, and a CDR3 of the amino acid sequence of SEQ ID NO: 56; and
a light chain variable region comprising: a CDR1, a CDR2, and a CDR3 amino acid sequences of SEQ ID NO: 8;
xii. a heavy chain variable region comprising: a CDR1, a CDR2, and a CDR3 of the amino acid sequence of SEQ ID NO: 60; and
a light chain variable region comprising: a CDR1, a CDR2, and a CDR3 amino acid sequences of SEQ ID NO: 8;
xiii. a heavy chain variable region comprising: a CDR1, a CDR2, and a CDR3 of the amino acid sequence of SEQ ID NO: 64; and
a light chain variable region comprising: a CDR1, a CDR2, and a CDR3 amino acid sequences of SEQ ID NO: 8;
xiv. a heavy chain variable region comprising: a CDR1, a CDR2, and a CDR3 of the amino acid sequence of SEQ ID NO: 68; and
a light chain variable region comprising: a CDR1, a CDR2, and a CDR3 amino acid sequences of SEQ ID NO: 8;
xv. a heavy chain variable region comprising: a CDR1, a CDR2, and a CDR3 of the amino acid sequence of SEQ ID NO: 72; and
a light chain variable region comprising: a CDR1, a CDR2, and a CDR3 amino acid sequences of SEQ ID NO: 8;
xvi. a heavy chain variable region comprising: a CDR1, a CDR2, and a CDR3 of the amino acid sequence of SEQ ID NO: 76; and
a light chain variable region comprising: a CDR1, a CDR2, and a CDR3 amino acid sequences of SEQ ID NO: 8; or
xvii. a heavy chain variable region comprising: a CDR1, a CDR2, and a CDR3 of the amino acid sequence of SEQ ID NO: 80; and
a light chain variable region comprising: a CDR1, a CDR2, and a CDR3 amino acid sequences of SEQ ID NO: 8; and
under conditions that allow for formation of a complex between the antibody or antigen-binding fragment thereof and GPC3 present in the sample, and measuring a detectable signal associated with the formation of the complex.

27. The method according to claim 26, wherein the antibody or antigen-binding fragment thereof of claim 1, comprises:
i. a heavy chain variable region comprising SEQ ID NO: 1; and
a light chain variable region comprising SEQ ID NO: 2;
ii. a heavy chain variable region comprising SEQ ID NO: 3; and
a light chain variable region comprising SEQ ID NO: 4;
iii. a heavy chain variable region comprising SEQ ID NO: 5; and
a light chain variable region comprising SEQ ID NO: 6;
iv. a heavy chain variable region comprising SEQ ID NO: 7; and
a light chain variable region comprising SEQ ID NO: 8;
v. a heavy chain variable region comprising SEQ ID NO: 9; and
a light chain variable region comprising SEQ ID NO: 10;
vi. a heavy chain variable region comprising SEQ ID NO: 11; and
a light chain variable region comprising SEQ ID NO: 12;
vii. a heavy chain variable region comprising SEQ ID NO: 13; and
a light chain variable region comprising SEQ ID NO: 14;
viii. a heavy chain variable region comprising SEQ ID NO: 15; and
a light chain variable region comprising SEQ ID NO: 16;
ix. a heavy chain variable region comprising SEQ ID NO: 48; and
a light chain variable region comprising SEQ ID NO: 8;
x. a heavy chain variable region comprising SEQ ID NO: 52; and
a light chain variable region comprising SEQ ID NO: 8;
xi. a heavy chain variable region comprising SEQ ID NO: 56; and
a light chain variable region comprising SEQ ID NO: 8;
xii. a heavy chain variable region comprising SEQ ID NO: 60; and
a light chain variable region comprising SEQ ID NO: 8;
xiii. a heavy chain variable region comprising SEQ ID NO: 64; and
a light chain variable region comprising SEQ ID NO: 8;
xiv. a heavy chain variable region comprising SEQ ID NO: 68; and
a light chain variable region comprising SEQ ID NO: 8;
xv. a heavy chain variable region comprising SEQ ID NO: 72; and
a light chain variable region comprising SEQ ID NO: 8;
xvi. a heavy chain variable region comprising SEQ ID NO: 76; and
a light chain variable region comprising SEQ ID NO: 8; or
xvii. a heavy chain variable region comprising SEQ ID NO: 80; and
a light chain variable region comprising SEQ ID NO: 8.

28. The method according to any one of claims 26-27, wherein the antibody or antigen-binding fragment thereof comprises a detectable label.

29. The method of any one of claims 18-21, wherein the subject is human.

30. The method of any one of claims 22-25, wherein the cell is a human cell.

31. The method of any one of claims 26-28, wherein the biological sample is from a human.

32. An isolated polynucleotide encoding an antibody or antigen-binding fragment thereof that binds to glypican 3 (GPC3) protein, wherein the antibody or antigen-binding fragment thereof comprises:
i. a heavy chain variable region comprising: a CDR1, a CDR2, and a CDR3 of the amino acid sequence of SEQ ID NO: 1; and
a light chain variable region comprising: a CDR1, a CDR2, and a CDR3 amino acid sequences of SEQ ID NO: 2;
ii. a heavy chain variable region comprising: a CDR1, a CDR2, and a CDR3 of the amino acid sequence of SEQ ID NO: 3; and
a light chain variable region comprising: a CDR1, a CDR2, and a CDR3 amino acid sequences of SEQ ID NO: 4;
iii. a heavy chain variable region comprising: a CDR1, a CDR2, and a CDR3 of the amino acid sequence of SEQ ID NO: 5; and
a light chain variable region comprising: a CDR1, a CDR2, and a CDR3 amino acid sequences of SEQ ID NO: 6;
iv. a heavy chain variable region comprising: a CDR1, a CDR2, and a CDR3 of the amino acid sequence of SEQ ID NO: 7; and
a light chain variable region comprising: a CDR1, a CDR2, and a CDR3 amino acid sequences of SEQ ID NO: 8;
v. a heavy chain variable region comprising: a CDR1, a CDR2, and a CDR3 of the amino acid sequence of SEQ ID NO: 9; and
a light chain variable region comprising: a CDR1, a CDR2, and a CDR3 amino acid sequences of SEQ ID NO: 10;
vi. a heavy chain variable region comprising: a CDR1, a CDR2, and a CDR3 of the amino acid sequence of SEQ ID NO: 11; and
a light chain variable region comprising: a CDR1, a CDR2, and a CDR3 amino acid sequences of SEQ ID NO: 12;
vii. a heavy chain variable region comprising: a CDR1, a CDR2, and a CDR3 of the amino acid sequence of SEQ ID NO: 13; and
a light chain variable region comprising: a CDR1, a CDR2, and a CDR3 amino acid sequences of SEQ ID NO: 14;
viii. a heavy chain variable region comprising: a CDR1, a CDR2, and a CDR3 of the amino acid sequence of SEQ ID NO: 15; and
a light chain variable region comprising: a CDR1, a CDR2, and a CDR3 amino acid sequences of SEQ ID NO: 16;
ix. a heavy chain variable region comprising: a CDR1, a CDR2, and a CDR3 of the amino acid sequence of SEQ ID NO: 48; and
a light chain variable region comprising: a CDR1, a CDR2, and a CDR3 amino acid sequences of SEQ ID NO: 8;
x. a heavy chain variable region comprising: a CDR1, a CDR2, and a CDR3 of the amino acid sequence of SEQ ID NO: 52; and
a light chain variable region comprising: a CDR1, a CDR2, and a CDR3 amino acid sequences of SEQ ID NO: 8;
xi. a heavy chain variable region comprising: a CDR1, a CDR2, and a CDR3 of the amino acid sequence of SEQ ID NO: 56; and
a light chain variable region comprising: a CDR1, a CDR2, and a CDR3 amino acid sequences of SEQ ID NO: 8;
xii. a heavy chain variable region comprising: a CDR1, a CDR2, and a CDR3 of the amino acid sequence of SEQ ID NO: 60; and
a light chain variable region comprising: a CDR1, a CDR2, and a CDR3 amino acid sequences of SEQ ID NO: 8;
xiii. a heavy chain variable region comprising: a CDR1, a CDR2, and a CDR3 of the amino acid sequence of SEQ ID NO: 64; and
a light chain variable region comprising: a CDR1, a CDR2, and a CDR3 amino acid sequences of SEQ ID NO: 8;
xiv. a heavy chain variable region comprising: a CDR1, a CDR2, and a CDR3 of the amino acid sequence of SEQ ID NO: 68; and
a light chain variable region comprising: a CDR1, a CDR2, and a CDR3 amino acid sequences of SEQ ID NO: 8;
xv. a heavy chain variable region comprising: a CDR1, a CDR2, and a CDR3 of the amino acid sequence of SEQ ID NO: 72; and
a light chain variable region comprising: a CDR1, a CDR2, and a CDR3 amino acid sequences of SEQ ID NO: 8;
xvi. a heavy chain variable region comprising: a CDR1, a CDR2, and a CDR3 of the amino acid sequence of SEQ ID NO: 76; and
a light chain variable region comprising: a CDR1, a CDR2, and a CDR3 amino acid sequences of SEQ ID NO: 8; or
xvii. a heavy chain variable region comprising: a CDR1, a CDR2, and a CDR3 of the amino acid sequence of SEQ ID NO: 80; and
xviii. a light chain variable region comprising: a CDR1, a CDR2, and a CDR3 amino acid sequences of SEQ ID NO: 8

33. The isolated polynucleotide of claim 32, wherein the antibody or antigen-binding fragment thereof comprises:
i. a heavy chain variable region comprising SEQ ID NO: 1; and
a light chain variable region comprising SEQ ID NO: 2;
ii. a heavy chain variable region comprising SEQ ID NO: 3; and
a light chain variable region comprising SEQ ID NO: 4;
iii. a heavy chain variable region comprising SEQ ID NO: 5; and
a light chain variable region comprising SEQ ID NO: 6;
iv. a heavy chain variable region comprising SEQ ID NO: 7; and
a light chain variable region comprising SEQ ID NO: 8;
v. a heavy chain variable region comprising SEQ ID NO: 9; and
a light chain variable region comprising SEQ ID NO: 10;
vi. a heavy chain variable region comprising SEQ ID NO: 11; and
a light chain variable region comprising SEQ ID NO: 12;
vii. a heavy chain variable region comprising SEQ ID NO: 13; and
a light chain variable region comprising SEQ ID NO: 14;
viii. a heavy chain variable region comprising SEQ ID NO: 15; and
a light chain variable region comprising SEQ ID NO: 16;
ix. a heavy chain variable region comprising SEQ ID NO: 48; and
a light chain variable region comprising SEQ ID NO: 8;
x. a heavy chain variable region comprising SEQ ID NO: 52; and
a light chain variable region comprising SEQ ID NO: 8;
xi. a heavy chain variable region comprising SEQ ID NO: 56; and
a light chain variable region comprising SEQ ID NO: 8;
xii. a heavy chain variable region comprising SEQ ID NO: 60; and
a light chain variable region comprising SEQ ID NO: 8;
xiii. a heavy chain variable region comprising SEQ ID NO: 64; and
a light chain variable region comprising SEQ ID NO: 8;
xiv. a heavy chain variable region comprising SEQ ID NO: 68; and
a light chain variable region comprising SEQ ID NO: 8;
xv. a heavy chain variable region comprising SEQ ID NO: 72; and
a light chain variable region comprising SEQ ID NO: 8;
xvi. a heavy chain variable region comprising SEQ ID NO: 76; and
a light chain variable region comprising SEQ ID NO: 8; or
xvii. a heavy chain variable region comprising SEQ ID NO: 80; and
a light chain variable region comprising SEQ ID NO: 8.

34. An isolated recombinant cell that produces an antibody or antigen-binding fragment thereof that binds to glypican 3 (GPC3) protein wherein the antibody or antigen-binding fragment thereof comprises:
i. a heavy chain variable region comprising: a CDR1, a CDR2, and a CDR3 of the amino acid sequence of SEQ ID NO: 1; and
a light chain variable region comprising: a CDR1, a CDR2, and a CDR3 amino acid sequences of SEQ ID NO: 2;
ii. a heavy chain variable region comprising: a CDR1, a CDR2, and a CDR3 of the amino acid sequence of SEQ ID NO: 3; and
a light chain variable region comprising: a CDR1, a CDR2, and a CDR3 amino acid sequences of SEQ ID NO: 4;
iii. a heavy chain variable region comprising: a CDR1, a CDR2, and a CDR3 of the amino acid sequence of SEQ ID NO: 5; and
a light chain variable region comprising: a CDR1, a CDR2, and a CDR3 amino acid sequences of SEQ ID NO: 6;
iv. a heavy chain variable region comprising: a CDR1, a CDR2, and a CDR3 of the amino acid sequence of SEQ ID NO: 7; and
a light chain variable region comprising: a CDR1, a CDR2, and a CDR3 amino acid sequences of SEQ ID NO: 8;
v. a heavy chain variable region comprising: a CDR1, a CDR2, and a CDR3 of the amino acid sequence of SEQ ID NO: 9; and
a light chain variable region comprising: a CDR1, a CDR2, and a CDR3 amino acid sequences of SEQ ID NO: 10;
vi. a heavy chain variable region comprising: a CDR1, a CDR2, and a CDR3 of the amino acid sequence of SEQ ID NO: 11; and
a light chain variable region comprising: a CDR1, a CDR2, and a CDR3 amino acid sequences of SEQ ID NO: 12;
vii. a heavy chain variable region comprising: a CDR1, a CDR2, and a CDR3 of the amino acid sequence of SEQ ID NO: 13; and
a light chain variable region comprising: a CDR1, a CDR2, and a CDR3 amino acid sequences of SEQ ID NO: 14;
viii. a heavy chain variable region comprising: a CDR1, a CDR2, and a CDR3 of the amino acid sequence of SEQ ID NO: 15; and
a light chain variable region comprising: a CDR1, a CDR2, and a CDR3 amino acid sequences of SEQ ID NO: 16;
ix. a heavy chain variable region comprising: a CDR1, a CDR2, and a CDR3 of the amino acid sequence of SEQ ID NO: 48; and
a light chain variable region comprising: a CDR1, a CDR2, and a CDR3 amino acid sequences of SEQ ID NO: 8;
x. a heavy chain variable region comprising: a CDR1, a CDR2, and a CDR3 of the amino acid sequence of SEQ ID NO: 52; and
a light chain variable region comprising: a CDR1, a CDR2, and a CDR3 amino acid sequences of SEQ ID NO: 8;
xi. a heavy chain variable region comprising: a CDR1, a CDR2, and a CDR3 of the amino acid sequence of SEQ ID NO: 56; and
a light chain variable region comprising: a CDR1, a CDR2, and a CDR3 amino acid sequences of SEQ ID NO: 8;
xii. a heavy chain variable region comprising: a CDR1, a CDR2, and a CDR3 of the amino acid sequence of SEQ ID NO: 60; and
a light chain variable region comprising: a CDR1, a CDR2, and a CDR3 amino acid sequences of SEQ ID NO: 8;
xiii. a heavy chain variable region comprising: a CDR1, a CDR2, and a CDR3 of the amino acid sequence of SEQ ID NO: 64; and
a light chain variable region comprising: a CDR1, a CDR2, and a CDR3 amino acid sequences of SEQ ID NO: 8;
xiv. a heavy chain variable region comprising: a CDR1, a CDR2, and a CDR3 of the amino acid sequence of SEQ ID NO: 68; and
a light chain variable region comprising: a CDR1, a CDR2, and a CDR3 amino acid sequences of SEQ ID NO: 8;
xv. a heavy chain variable region comprising: a CDR1, a CDR2, and a CDR3 of the amino acid sequence of SEQ ID NO: 72; and
a light chain variable region comprising: a CDR1, a CDR2, and a CDR3 amino acid sequences of SEQ ID NO: 8;
xvi. a heavy chain variable region comprising: a CDR1, a CDR2, and a CDR3 of the amino acid sequence of SEQ ID NO: 76; and
a light chain variable region comprising: a CDR1, a CDR2, and a CDR3 amino acid sequences of SEQ ID NO: 8; or
xvii. a heavy chain variable region comprising: a CDR1, a CDR2, and a CDR3 of the amino acid sequence of SEQ ID NO: 80; and
xviii. a light chain variable region comprising: a CDR1, a CDR2, and a CDR3 amino acid sequences of SEQ ID NO: 8

35. The isolated polynucleotide of claim 34, wherein the antibody or antigen-binding fragment thereof comprises:
i. a heavy chain variable region comprising SEQ ID NO: 1; and
a light chain variable region comprising SEQ ID NO: 2;
ii. a heavy chain variable region comprising SEQ ID NO: 3; and
a light chain variable region comprising SEQ ID NO: 4;
iii. a heavy chain variable region comprising SEQ ID NO: 5; and
a light chain variable region comprising SEQ ID NO: 6;
iv. a heavy chain variable region comprising SEQ ID NO: 7; and
a light chain variable region comprising SEQ ID NO: 8;
v. a heavy chain variable region comprising SEQ ID NO: 9; and
a light chain variable region comprising SEQ ID NO: 10;
vi. a heavy chain variable region comprising SEQ ID NO: 11; and
a light chain variable region comprising SEQ ID NO: 12;
vii. a heavy chain variable region comprising SEQ ID NO: 13; and
a light chain variable region comprising SEQ ID NO: 14;
viii. a heavy chain variable region comprising SEQ ID NO: 15; and
a light chain variable region comprising SEQ ID NO: 16;
ix. a heavy chain variable region comprising SEQ ID NO: 48; and
a light chain variable region comprising SEQ ID NO: 8;
x. a heavy chain variable region comprising SEQ ID NO: 52; and
a light chain variable region comprising SEQ ID NO: 8;
xi. a heavy chain variable region comprising SEQ ID NO: 56; and
a light chain variable region comprising SEQ ID NO: 8;
xii. a heavy chain variable region comprising SEQ ID NO: 60; and
a light chain variable region comprising SEQ ID NO: 8;
xiii. a heavy chain variable region comprising SEQ ID NO: 64; and
a light chain variable region comprising SEQ ID NO: 8;
xiv. a heavy chain variable region comprising SEQ ID NO: 68; and
a light chain variable region comprising SEQ ID NO: 8;
xv. a heavy chain variable region comprising SEQ ID NO: 72; and
a light chain variable region comprising SEQ ID NO: 8;
xvi. a heavy chain variable region comprising SEQ ID NO: 76; and
a light chain variable region comprising SEQ ID NO: 8; or
xvii. a heavy chain variable region comprising SEQ ID NO: 80; and
a light chain variable region comprising SEQ ID NO: 8.


SEQUENCE LISTING (102 sequences)

SEQ ID NO: 1 - protein, 119 aa, Synthetic construct
QVQLQQSGPGLVKPSQTLSLTCAISGDSVSSYSAAWNWIRQSPSRGLEWLGRTYYRSKWYNDYEASVKSRITFKADTSKNQLSLQLNSVTPEDTAVYYCARGQWRFDYWGQGTLVTVSS

SEQ ID NO: 2 - protein, 108 aa, Synthetic construct
SYELTQPPSVAVAPGQTARITCEEYNIESKSVHWYQEKPGQAPVLVIYNDGDRPSGVPERFSGSNSGNTATLTINRVEAGDEAEYYCXAWDIGTDHPIFGVGTKLTVL

SEQ ID NO: 3 - protein, 122 aa, Synthetic construct
QVQLQQSGPGLVKPSQTLSITCAISGDSVSSNSAAWNWIRQSPSRGLEWLGRTYYRSKWYNDYAVSVKSRITINPDTSKNQFSLQLNSVTPEDTAVYYCASSYYNGGYFDYWGQGTLVTVSS

SEQ ID NO: 4 - protein, 106 aa, Synthetic construct
EIVLTQSPATLSVSPGGRATLSCRASQSVTTNLAWYQQKPGQAPRLLIYGASTRVTGVPRRFSGSGSGTDFTLTISSLQSEDSAVYYCQQYSGWGGFGGGTKVDIK

SEQ ID NO: 5 - protein, 120 aa, Synthetic construct
EVQLVQSGAEVKKPGSSVKVSCKASGGTFRSYAISWVRQAPGQGLEWMGGIIPIFGTANYAQKFQGRVTITADELTSTAYMELSSLRSEDTAVYYCASRIGQWPTPFDYWGQGTLVTVSS

SEQ ID NO: 6 - protein, 108 aa, Synthetic construct
QSVLTQPPSVSVAPGKTARITCGGNNIGSKSVHWYQQKPGQAPVLVIYYDSDRPSGIPERFSGSNSGNTATLTISRVEAGDEADYYCQVWDSSSDHPVFGGGTKLTVL

SEQ ID NO: 7 - protein, 126 aa, Synthetic construct
EVQLVESGGGLVQPGGSLRLSCAASGFTFRSYAMSWVRQAPGKGLEWVSAISGSGGRTYYADSVKGRFTISRDNSKNTLYLQMNSLRAEDTAVYYCAKEAPYSSGWSHYYYGMDVWGQGTTVTVSS

SEQ ID NO: 8 - protein, 106 aa, Synthetic construct
QSVLTQPPSVSGAPGQRVTISCTGSSSGYDVHWYQQLPGRAPKLLIYGSNNRPSGVPDRFSGSNSGTSASLAITGLQAEDEADYYCQSYDSSLNAIFGGGTKLTVL

SEQ ID NO: 9 - protein, 114 aa, Synthetic construct
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCASVYMDVWGKGTTVTVSS

SEQ ID NO: 10 - protein, 108 aa, Synthetic construct
EIVMTQSPGTLSLSPGERATLSCRASQTVSSSSLAWYQQRPGQAPRLLIYETSSRATGIPDRFSGSGSGTDFTLTISRLEPEDFAIYYCQQKGDSGYTFGQGTKVEIK

SEQ ID NO: 11 - protein, 114 aa, Synthetic construct; identical to SEQ ID NO: 9

SEQ ID NO: 12 - protein, 108 aa, Synthetic construct
SYELTQPPSVSVAPGQTARITCGGNNIGSKSVHWYQQKPGQAPVLVVYDDSDRPSGVPERFSGSNSGNTATLTISRVEAGDEADYYCQVWDNSSDHQVFGGGTKLTVL

SEQ ID NO: 13 - protein, 120 aa, Synthetic construct
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCARASGRVTDAFDIWGQGTMVTVSS

SEQ ID NO: 14 - protein, 115 aa, Synthetic construct
QAVLTQPSSLSASPGASASLTCTLRSGINVVPYRIYWYQQKPGSPPQFLLNYRSDSDNYQGSGVPSRFSGSKDTSANAGILLISGLQSEDEADYYCLTWHSDSWVFGGGTKLTVL

SEQ ID NO: 15 - protein, 120 aa, Synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGGDGYNAAFDIWGQGTMVTVSS

SEQ ID NO: 16 - protein, 111 aa, Synthetic construct
NFMLTQPPSVSESPGKTVTISCTGSSGSIASNYVQWYQQRPGSAPTTVIYEDNQRPSGVPDRFSGSIDSSSNSASLTISGLKTEDEADYYCQSYDSSKHYVFGTGTKVTVL

SEQ ID NO: 17 - DNA, 42 bp, Synthetic construct
  source 1..42: mol_type = other DNA
gcctctnhsnhsnhsnhsnhsagctatgccatgagctgggtc

SEQ ID NO: 18 - DNA, 22 bp, Synthetic construct
  source 1..22: mol_type = other DNA
tgcacaggagagtctcagggac

SEQ ID NO: 19 - DNA, 39 bp, Synthetic construct
  source 1..39: mol_type = other DNA
ttcaccnhsnhsnhsnhsnhsatgagctgggtccgccag

SEQ ID NO: 20 - DNA, 19 bp, Synthetic construct
  source 1..19: mol_type = other DNA
tccagaggctgcacaggag

SEQ ID NO: 21 - DNA, 48 bp, Synthetic construct
  source 1..48: mol_type = other DNA
tcagctnhsnhsnhsnhsnhsggtagaacatactacgcagactccgtg

SEQ ID NO: 22 - DNA, 20 bp, Synthetic construct
  source 1..20: mol_type = other DNA
gacccactccagccccttcc

SEQ ID NO: 23 - DNA, 44 bp, Synthetic construct
  source 1..44: mol_type = other DNA
agtggtnhsnhsnhsnhsnhstactacgcagactccgtgaaggg

SEQ ID NO: 24 - DNA, 22 bp, Synthetic construct
  source 1..22: mol_type = other DNA
aatagctgagacccactccagc

SEQ ID NO: 25 - DNA, 46 bp, Synthetic construct
  source 1..46: mol_type = other DNA
gcgaaanhsnhsnhsnhsnhsagtggctggtctcactactactacg

SEQ ID NO: 26 - DNA, 47 bp, Synthetic construct
  source 1..47: mol_type = other DNA
tatagcnhsnhsnhsnhsnhstactactacggtatggacgtctgggg

SEQ ID NO: 27 - DNA, 19 bp, Synthetic construct
  source 1..19: mol_type = other DNA
cggggcttctttcgcacag

SEQ ID NO: 28 - DNA, 40 bp, Synthetic construct
  source 1..40: mol_type = other DNA
tggtctnhsnhsnhsnhsnhsatggacgtctggggccaag

SEQ ID NO: 29 - DNA, 21 bp, Synthetic construct
  source 1..21: mol_type = other DNA
gccactgctatacggggcttc

SEQ ID NO: 30 - DNA, 44 bp, Synthetic construct
  source 1..44: mol_type = other DNA
gggagcnhsnhsnhsnhsnhsgtacactggtaccagcagcttcc

SEQ ID NO: 31 - DNA, 22 bp, Synthetic construct
  source 1..22: mol_type = other DNA
agtgcaggagatggtgaccctc

SEQ ID NO: 32 - DNA, 33 bp, Synthetic construct
  source 1..33: mol_type = other DNA
atctatnhsnhsnhsaatcggccctcaggggtc

SEQ ID NO: 33 - DNA, 23 bp, Synthetic construct
  source 1..23: mol_type = other DNA
gaggagtttgggggctcttcctg

SEQ ID NO: 34 - DNA, 46 bp, Synthetic construct
  source 1..46: mol_type = other DNA
tactgcnhsnhsnhsnhsnhsagcctgaatgctatattcggcggag

SEQ ID NO: 35 - DNA, 25 bp, Synthetic construct
  source 1..25: mol_type = other DNA
ataatcagcctcatcctcagcctgg

SEQ ID NO: 36 - DNA, 42 bp, Synthetic construct
  source 1..42: mol_type = other DNA
tcctatnhsnhsnhsnhsnhsgctatattcggcggagggacc

SEQ ID NO: 37 - DNA, 27 bp, Synthetic construct
  source 1..27: mol_type = other DNA
ctggcagtaataatcagcctcatcctc

SEQ ID NO: 38 - DNA, 49 bp, Synthetic construct
  source 1..49: mol_type = other DNA
gcagcctctggattcacctttrnsrvwtatgncatgagctgggtccgcc

SEQ ID NO: 39 - DNA, 22 bp, Synthetic construct
  source 1..22: mol_type = other DNA
tgagacccactccagccccttc

SEQ ID NO: 40 - DNA, 60 bp, Synthetic construct
  source 1..60: mol_type = other DNA
gggctggagtgggtctcarntntsagtrvtagtggtrntrnsacatactacgcagactcc

SEQ ID NO: 41 - DNA, 27 bp, Synthetic construct
  source 1..27: mol_type = other DNA
cccattcagatcctcttctgagatgag

SEQ ID NO: 42 - DNA, 42 bp, Synthetic construct
  source 1..42: mol_type = other DNA
aaagawnnsnnsntsnnsnnsggctggtctcactactactac

SEQ ID NO: 43 - DNA, 22 bp, Synthetic construct
  source 1..22: mol_type = other DNA
cgcacagtaatatacggccgtg

SEQ ID NO: 44 - DNA, 42 bp, Synthetic construct
  source 1..42: mol_type = other DNA
tatagcnnsngsnnsnnsnnstactactacggtatggacgtc

SEQ ID NO: 45 - DNA, 27 bp, Synthetic construct
  source 1..27: mol_type = other DNA
cggggcttctttcgcacagtaatatac

SEQ ID NO: 46 - DNA, 56 bp, Synthetic construct
  source 1..56: mol_type = other DNA
ccgtatattactgtgcgaaagawnnsgactwcnnsnnsggctggwygcactactac

SEQ ID NO: 47 - DNA, 27 bp, Synthetic construct
  source 1..27: mol_type = other DNA
cccattcagatcctcttctgagatgag

SEQ ID NO: 48 - protein, 126 aa, Synthetic construct
EVQLVESGGGLVQPGGSLRLSCAASGFTFRSYAMSWVRQAPGKGLEWVSAISGSGGRTYYADSVKGRFTISRDNSKNTLYLQMNSLRAEDTAVYYCAKEYDFKTGWMHYYYGMDVWGQGTTVTVSS

SEQ ID NO: 49 - protein, 8 aa, Synthetic construct
GFTFRSYA

SEQ ID NO: 50 - protein, 8 aa, Synthetic construct
ISGSGGRT

SEQ ID NO: 51 - protein, 19 aa, Synthetic construct
AKEYDFKTGWMHYYYGMDV

SEQ ID NO: 52 - protein, 126 aa, Synthetic construct
EVQLVESGGGLVQPGGSLRLSCAASGFTFKDYAMSWVRQAPGKGLEWVSALSGSGGATYYADSVKGRFTISRDNSKNTLYLQMNSLRAEDTAVYYCAKEMDFRYGWMHYYYGMDVWGQGTTVTVSS

SEQ ID NO: 53 - protein, 8 aa, Synthetic construct
GFTFKDYA

SEQ ID NO: 54 - protein, 8 aa, Synthetic construct
LSGSGGAT

SEQ ID NO: 55 - protein, 19 aa, Synthetic construct
AKEMDFRYGWMHYYYGMDV

SEQ ID NO: 56 - protein, 126 aa, Synthetic construct
EVQLVESGGGLVQPGGSLRLSCAASGFTFEGYAMSWVRQAPGKGLEWVSNVSGSGDGTYYADSVKGRFTISRDNSKNTLYLQMNSLRAEDTAVYYCAKEYDYKTGWMHYYYGMDVWGQGTTVTVSS

SEQ ID NO: 57 - protein, 8 aa, Synthetic construct
GFTFEGYA

SEQ ID NO: 58 - protein, 8 aa, Synthetic construct
VSGSGDGT

SEQ ID NO: 59 - protein, 19 aa, Synthetic construct
AKEYDYKTGWMHYYYGMDV

SEQ ID NO: 60 - protein, 126 aa, Synthetic construct
EVQLVESGGGLVQPGGSLRLSCAASGFTFGEYAMSWVRQAPGKGLEWVSGMSGSGDGTYYADSVKGRFTISRDNSKNTLYLQMNSLRAEDTAVYYCAKEMDYQLGWMHYYYGMDVWGQGTTVTVSS

SEQ ID NO: 61 - protein, 8 aa, Synthetic construct
GFTFGEYA

SEQ ID NO: 62 - protein, 8 aa, Synthetic construct
MSGSGDGT

SEQ ID NO: 63 - protein, 19 aa, Synthetic construct
AKEMDYQLGWMHYYYGMDV

SEQ ID NO: 64 - protein, 126 aa, Synthetic construct
EVQLVESGGGLVQPGGSLRLSCAASGFTFANYAMSWVRQAPGKGLEWVSDVSASGVGTYYADSVKGRFTISRDNSKNTLYLQMNSLRAEDTAVYYCAKEVDFKLGWMHYYYGMDVWGQGTTVTVSS

SEQ ID NO: 65 - protein, 8 aa, Synthetic construct
GFTFANYA

SEQ ID NO: 66 - protein, 8 aa, Synthetic construct
VSASGVGT

SEQ ID NO: 67 - protein, 19 aa, Synthetic construct
AKEVDFKLGWMHYYYGMDV

SEQ ID NO: 68 - protein, 126 aa, Synthetic construct
EVQLVESGGGLVQPGGSLRLSCAASGFTFGNYAMSWVRQAPGKGLEWVSGVSGSGDGTYYADSVKGRFTISRDNSKNTLYLQMNSLRAEDTAVYYCAKEYDFGLGWMHYYYGMDVWGQGTTVTVSS

SEQ ID NO: 69 - protein, 8 aa, Synthetic construct
GFTFGNYA

SEQ ID NO: 70 - protein, 8 aa, Synthetic construct
VSGSGDGT

SEQ ID NO: 71 - protein, 19 aa, Synthetic construct
AKEYDFGLGWMHYYYGMDV

SEQ ID NO: 72 - protein, 126 aa, Synthetic construct
EVQLVESGGGLVQPGGSLRLSCAASGFTFKRYAMSWVRQAPGKGLEWVSSVSSSGTGTYYADSVKGRFTISRDNSKNTLYLQMNSLRAEDTAVYYCAKEYDYGLGWMHYYYGMDVWGQGTTVTVSS

SEQ ID NO: 73 - protein, 8 aa, Synthetic construct
GFTFKRYA

SEQ ID NO: 74 - protein, 8 aa, Synthetic construct
VSSSGTGT

SEQ ID NO: 75 - protein, 19 aa, Synthetic construct
AKEYDYGLGWMHYYYGMDV

SEQ ID NO: 76 - protein, 126 aa, Synthetic construct
EVQLVESGGGLVQPGGSLRLSCAASGFTFDRYAMSWVRQAPGKGLEWVSSLSASGSGTYYADSVKGRFTISRDNSKNTLYLQMNSLRAEDTAVYYCAKEYDFSYGWMHYYYGMDVWGQGTTVTVSS

SEQ ID NO: 77 - protein, 8 aa, Synthetic construct
GFTFDRYA

SEQ ID NO: 78 - protein, 8 aa, Synthetic construct
LSASGSGT

SEQ ID NO: 79 - protein, 19 aa, Synthetic construct
AKEYDFSYGWMHYYYGMDV

SEQ ID NO: 80 - protein, 126 aa, Synthetic construct
EVQLVESGGGLVQPGGSLRLSCAASGFTFRKYAMSWVRQAPGKGLEWVSSLSASGSATYYADSVKGRFTISRDNSKNTLYLQMNSLRAEDTAVYYCAKEADYSMGWMHYYYGMDVWGQGTTVTVSS

SEQ ID NO: 81 - protein, 13 aa, Synthetic construct
GFTFRKYASEQID

SEQ ID NO: 82 - protein, 8 aa, Synthetic construct
LSASGSAT

SEQ ID NO: 83 - protein, 19 aa, Synthetic construct
AKEADYSMGWMHYYYGMDV

SEQ ID NO: 84 - protein, 580 aa, Synthetic construct
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 85 - protein, 5 aa, Synthetic construct
GFTFR

SEQ ID NO: 86 - protein, 5 aa, Synthetic construct
FRSYA

SEQ ID NO: 87 - protein, 5 aa, Synthetic construct
ISGSG

SEQ ID NO: 88 - protein, 5 aa, Synthetic construct
SGGRT

SEQ ID NO: 89 - protein, 5 aa, Synthetic construct
EAPYS

SEQ ID NO: 90 - protein, 5 aa, Synthetic construct
SGWSH

SEQ ID NO: 91 - protein, 5 aa, Synthetic construct
HYYYG

SEQ ID NO: 92 - DNA, 25 bp, Synthetic construct
  source 1..25: mol_type = other DNA
acagtaatatacggccgtgtcctcg

SEQ ID NO: 93 - protein, 5 aa, Synthetic construct
SSGYD

SEQ ID NO: 94 - skipped

SEQ ID NO: 95 - protein, 5 aa, Synthetic construct
QSYDS

SEQ ID NO: 96 - protein, 5 aa, Synthetic construct
DSSLN

SEQ ID NO: 97 - protein, 4 aa, Synthetic construct
RSYA

SEQ ID NO: 98 - protein, 8 aa, Synthetic construct
AISGSGGR

SEQ ID NO: 99 - protein, 6 aa, Synthetic construct
EAPYSS

SEQ ID NO: 100 - protein, 5 aa, Synthetic construct
SGWSH

SEQ ID NO: 101 - protein, 9 aa, Synthetic construct
EAPYSSGWS

SEQ ID NO: 102 - DNA, 42 bp, Synthetic construct; identical to SEQ ID NO: 17
  source 1..42: mol_type = other DNA